(19) United States
(12) Patent Application Publication
(10) Pub. No.: US 2026/0238623 A1
(43) Pub. Date: Aug. 13, 2026

(54) SYSTEM AND METHOD FOR CREATING A TAMPER-PROOF AUDIT TRAIL FOR CONTAINER-MONITORING DATA

(71) Applicant: BARREL PROOF TECHNOLOGIES LLC, Murfreesboro, TN (US)

(72) Inventor: Brian Richard Anderson, Murfreesboro, TN (US)

(21) Appl. No.: 19/637,996

(22) Filed: Apr. 2, 2026

Related U.S. Application Data
(63) Continuation of application No. 19/636,514, filed on Apr. 1, 2026.
Application No. 19/636,514 is a continuation-in-part of application No. 19/532,912, filed on Feb. 6, 2026.
Application No. 19/532,912 is a continuation-in-part of application No. 19/241,167, filed on Jun. 17, 2025.
Application No. 19/241,167 is a continuation-in-part of application No. 19/235,377, filed on Jun. 11, 2025.
Application No. 19/235,377 is a continuation-in-part of application No. 19/182,441, filed on Apr. 17, 2025, now Pat. No. 12,669,362.
Application No. 19/235,377 is a continuation-in-part of application No. 19/084,671, filed on Mar. 19, 2025, now Pat. No. 12,609,185.
Application No. 19/182,441 is a continuation-in-part of application No. 19/080,723, filed on Mar. 14, 2025, now Pat. No. 12,422,295.
Application No. 19/080,723 is a continuation-in-part of application No. 19/013,859, filed on Jan. 8, 2025.
Application No. 19/084,671 is a continuation-in-part of application No. 19/013,859, filed on Jan. 8, 2025.
Application No. 19/013,859 is a continuation-in-part of application No. 18/818,539, filed on Aug. 28, 2024, now Pat. No. 12,228,525.
Application No. 19/013,859 is a continuation-in-part of application No. 18/800,279, filed on Aug. 12, 2024, now Pat. No. 12,228,445.
Application No. 18/800,279 is a continuation-in-part of application No. 18/424,758, filed on Jan. 27, 2024, now Pat. No. 12,117,329.
Application No. 18/818,539 is a continuation-in-part of application No. 18/424,758, filed on Jan. 27, 2024, now Pat. No. 12,117,329.

(51) Int. Cl. H04L 9/40

(52) U.S. Cl. CPC H04L 63/0428

(57) ABSTRACT

A method for creating a tamper-proof audit trail for container monitoring data includes acquiring sensor data from a noninvasive sensor monitoring a container, generating a cryptographic hash of the sensor data at an edge computing device, transmitting the sensor data and the cryptographic hash to a remote server over an encrypted communication channel, processing the sensor data using a machine learning model to generate a prediction regarding contents of the container, and recording the cryptographic hash on a distributed blockchain ledger to create an immutable record of the sensor data. In an embodiment, the method may further include constructing a dielectric spectrum based on the dielectric property measurement across the plurality of frequencies, comparing the dielectric spectrum to a baseline dielectric fingerprint, and detecting a contamination event based on a deviation between the dielectric spectrum and the baseline dielectric fingerprint exceeding a predefined threshold.

### CROSS-REFERENCE TO RELATED APPLICATIONS

This application is a Continuation of U.S. Application Number 19/636,514, titled SYSTEM AND METHOD FOR A UNIVERSAL, NONINVASIVE, MULTI-INDUSTRY INTEGRITY MONITORING AND CONTROL PLATFORM" filed on April 1, 2026, which is a Continuation-in-part of, and claims the benefit of U.S. Application Number 19/532,912, titled "EXTERNALLY POSITIONED FLUID LEVEL DETECTION AND SUMP PUMP CONTROL SYSTEM" filed on February 6, 2026, which is a Continuation-in-part of, and claims the benefit of U.S. Application Number 19/241,167, titled "NON-INVASIVE CONTAINER MONITORING USING A PASSIVE CERTIFICATION ELEMENT COUPLED TO A REMOVABLE SENSOR DEVICE" filed on June 17, 2025, which is a Continuation-in-part of, and claims the benefit of U. S. Application Number 19/235,377, titled "CONTAINER MONITORING SYSTEM WITH DIELECTRIC-BASED CONTAMINATION DETECTION" filed on June 11, 2025, which is a Continuation-in-part of, and claims the benefit of U. S. Application Number 19/182,441, titled "SYSTEM AND METHOD FOR DETERMINING CONTENT UTILIZING EXTERNALLY MOUNTED CONTAINER MONITORING SYSTEM" filed on April 17, 2025, which is a Continuation- in-part of, and claims the benefit of U. S. Application Number 19/080,723, titled "SYSTEM AND METHOD FOR DETERMINING CONTENT UTILIZING EXTERNALLY MOUNTED CONTAINER MONITORING SYSTEM" filed on March 14, 2025, which is a Continuation-in-part of, and claims the benefit of U. S. Application Number 19/013,859, titled "SYSTEM AND METHOD FOR DETERMINING FLUID LEVEL AND/OR ALCOHOL CONTENT UTILIZING EXTERNALLY MOUNTED CONTAINER MONITORING SYSTEM" filed on January 8, 2025, which is a Continuation-in-part of, and claims the benefit of U.S. Application Number 18/818,539, titled "SYSTEM AND METHOD FOR DETERMINING ALCOHOL CONTENT UTILIZING CONTAINER MONITORING SYSTEM," filed on August 28, 2024, which is a Continuation-in-part of, and claims the benefit and earlier filing date of U.S. Application Number 18/424,758, titled "CONTAINER MONITORING SYSTEM AND METHOD THEREOF," filed on January 27, 2024. U.S. Application Number 19/013,859 is also a continuation in part of, and claims the benefit of U.S. Application Number 18/800,279, titled "SYSTEM AND METHOD FOR DETERMINING ALCOHOL CONTENT WITHIN CONTAINER UTILIZING CONTAINER MONITORING SYSTEM," filed on August 12, 2024, which is a Continuation-in-part of, and claims the benefit and earlier filing date of U.S. Application Number 18/424,758, titled "CONTAINER MONITORING SYSTEM AND METHOD THEREOF," filed on January 27, 2024. This application incorporates by reference, herein, the entire contents of the above referred-to patent applications.

U.S. Application Number 19/235,377 is also a Continuation-in-part of, and claims the benefit of U.S. Application Number 19/084,671, titled "ARTIFICIAL INTELLIGENCE DRIVEN MONITORING SYSTEM FOR AGING WHISKEY" filed on March 19, 2025, which is a Continuation-in-part of, and claims the benefit of U. S. Application Number 19/013,859, titled "SYSTEM AND METHOD FOR DETERMINING FLUID LEVEL AND/OR ALCOHOL CONTENT UTILIZING EXTERNALLY MOUNTED CONTAINER MONITORING SYSTEM" filed on January 8, 2025, which is a Continuation-in-part of, and claims the benefit of U.S. Application Number 18/818,539, titled "SYSTEM AND METHOD FOR DETERMINING ALCOHOL CONTENT UTILIZING CONTAINER MONITORING SYSTEM," filed on August 28, 2024, which is a Continuation-in-part of, and claims the benefit and earlier filing date of U.S. Application Number 18/424,758, titled "CONTAINER MONITORING SYSTEM AND METHOD THEREOF," filed on January 27, 2024. U.S. Application Number 19/013,859 is also a continuation in part of, and claims the benefit of U.S. Application Number 18/800,279, titled "SYSTEM AND METHOD FOR DETERMINING ALCOHOL CONTENT WITHIN CONTAINER UTILIZING CONTAINER MONITORING SYSTEM," filed on August 12, 2024, which is a Continuation-in-part of, and claims the benefit and earlier filing date of U.S. Application Number 18/424,758, titled "CONTAINER MONITORING SYSTEM AND METHOD THEREOF," filed on January 27, 2024. This application incorporates by reference, herein, the entire contents of the above referred-to patent applications.

### TECHNICAL FIELD

The present disclosure relates to noninvasive sensing and monitoring systems, and more particularly to a software-defined platform for integrity monitoring and control of containerized assets across multiple industries using multi-modal sensor fusion, machine learning, digital twin simulation, and blockchain-enabled data verification.

### BACKGROUND

Industries that store, transport, or process liquids in containers face persistent challenges in monitoring the integrity, quality, and quantity of their contents. These industries span a wide range of applications, including spirits aging and distillation, military and commercial fuel logistics, pharmaceutical manufacturing and distribution, municipal and industrial water treatment, and residential and commercial flood prevention systems. Each of these domains involves containerized liquids where accurate, real-time monitoring of fill levels, composition, and contamination status can have substantial economic, safety, and regulatory implications.

Traditional approaches to container monitoring often rely on invasive sensing techniques that require physical penetration of the container wall or direct contact with the liquid contents. Such invasive methods can compromise container integrity, introduce potential contamination pathways, and may not be feasible for sealed containers such as aging barrels or pressurized tanks. Mechanical sensors, such as float switches used in sump pump applications, are prone to failure from debris accumulation, corrosion, and mechanical wear. Manual sampling and laboratory analysis, while accurate, provide only point-in-time snapshots and are expensive, time-consuming, and impractical for continuous monitoring of large inventories or distributed assets.

Noninvasive sensing technologies, including radar-based level measurement and electromagnetic characterization, offer alternatives that can measure container contents without physical contact. However, existing noninvasive systems are typically designed as purpose-built, vertically-integrated solutions optimized for a single application or industry. This fragmented approach results in separate hardware platforms for different use cases, limiting economies of scale in manufacturing and increasing complexity for organizations that operate across multiple domains.

The emergence of machine learning techniques has enabled more sophisticated interpretation of sensor data, allowing systems to extract meaningful information from complex signal patterns. Training such machine learning models, however, typically requires large quantities of labeled data that can be difficult and expensive to obtain in industrial settings. Collecting comprehensive datasets that cover all possible operating conditions, container types, liquid compositions, and contamination scenarios presents a substantial barrier to deploying machine learning-based monitoring systems.

Digital twin technology and physics-based simulation offer potential approaches for generating synthetic training data, but integrating such simulation capabilities with physical sensor systems and ensuring that synthetic data accurately represents real-world conditions remains challenging. Additionally, as monitoring systems generate increasing volumes of data, questions arise regarding data integrity, auditability, and the ability to provide verifiable records for regulatory compliance, insurance purposes, and financial transactions.

Distributed ledger technologies, including blockchain, have demonstrated capabilities for creating immutable records and enabling automated contract execution through smart contracts. The application of such technologies to industrial monitoring data could enable new business models and services, but integration with physical sensing systems and machine learning analytics presents technical challenges.

Accordingly, there exists a general interest in monitoring systems that can address the diverse needs of multiple industries while providing accurate, noninvasive sensing, intelligent data analysis, and verifiable data records.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

Herein disclosed is a universal, noninvasive integrity monitoring and control platform comprising a modular, externally-positioned sensor system and a software-defined architecture that enables a single hardware platform to be adapted for numerous industries by deploying different machine learning models. The platform employs a multi-modal sensor fusion approach, integrating Frequency Modulated Continuous Wave (FMCW) radar for fluid level detection and dielectric fingerprinting for content characterization and contamination detection. The device herein disclosed is applicable to a wide spectrum of configurations to monitor containerized assets, wherein the container may be associated with spirits aging and distillation, military and commercial fuel logistics, pharmaceutical manufacturing and distribution, municipal and industrial water treatment, residential and commercial flood prevention systems, or other types of materials or content, such as but not limited to, food and beverage storage, chemical processing, or environmental monitoring applications.

In accordance with further aspects of the invention, the disclosed monitoring system extends beyond traditional fluid level measurement to encompass comprehensive integrity assessment through multi-frequency dielectric fingerprinting. Each liquid substrate exhibits a unique electromagnetic signature based on its molecular composition, and by sweeping an RF or microwave signal across a range of frequencies and measuring the liquid's complex permittivity at each step, the system constructs a unique dielectric spectrum, or fingerprint, for the liquid. This fingerprint is highly sensitive to changes in composition, enabling the system to detect deviations indicative of contamination, tampering, degradation, dilution, substitution, or hazardous events. The specific shape and magnitude of spectral deviations may be used to classify contaminants, wherein water ingress causes a large, broad increase in permittivity, while other contaminants may cause more subtle, frequency- dependent shifts. This approach transforms passive storage containers into intelligent monitoring assets that can provide real-time alerts when liquid integrity is compromised.

The system architecture comprises modular sensor nodes that may be externally mounted to various container types without penetration or modification. These sensor nodes may incorporate an FMCW radar antenna for precise, noninvasive fluid level detection and a dielectric sensor array for electromagnetic interrogation of the container's contents. An edge computing module provides local processing for real-time machine learning inference and executes cryptographic hashing of sensor data at the point of collection to ensure a tamper- proof forensic metadata trail. A communication module provides flexible connectivity options and may support multi-node mesh networking. The measured dielectric signatures may be processed through industry-specific machine learning models that distinguish between normal variations and genuine integrity threats. A software-defined adaptation layer dynamically loads the appropriate machine learning model for a target industry, enabling the same hardware platform to serve diverse industries through model adaptation rather than equipment modification.

In some aspects, the Universal Hardware Platform 502 may accommodate sensing modalities beyond the FMCW Radar Antenna 520 and the Dielectric Sensor Array 522. The Modular Sensor Node 518 may define a standardized sensor abstraction interface through the Hot-Swap Sensor Interface 710 and the Auxiliary Sensor Ports 704 that enables the Edge Computing Module 524 to receive and process sensor data from any sensing modality through a common data format. In some aspects, the sensor abstraction interface may accept sensor data from one or more of an ultrasonic transducer configured to measure fluid level or material density through acoustic wave propagation, an optical sensor configured to measure absorption or scattering characteristics of the container contents through the container wall, an acoustic emission sensor configured to detect structural changes or fluid turbulence within the container, or an impedance spectroscopy sensor configured to measure the complex impedance of the container contents across a plurality of frequencies. Each alternative sensing modality may generate sensor data in a standardized format that the Edge Computing Module 524 processes using the loaded machine learning model, enabling the Software-Defined Adaptation Layer 506 to adapt to different sensing modalities as well as different industries. In some aspects, a single Modular Sensor Node 518 may incorporate two or more sensing modalities simultaneously, and the Synchronous Multi-Modal Integrity Verification 540 module may fuse data from any combination of available sensing modalities to perform integrity assessment. The standardized sensor abstraction interface may enable field upgradeability, where new sensing modalities developed after initial deployment may be added to existing sensor nodes without modification to the Edge Computing Module 524 or the Software-Defined Adaptation Layer 506.

The distributed monitoring platform integrates edge computing capabilities with cloud-based analytics to accommodate various deployment scenarios. In bandwidth- constrained or security-sensitive environments, sensor nodes may perform local inference using compressed and quantized machine learning models, providing autonomous operation without continuous connectivity. When network access is available, the system may synchronize detected anomalies and refined baselines with centralized infrastructure, enabling fleet-wide visibility and continuous model improvement through over-the-air model updates. In some aspects, the platform generates multi-tiered alerts ranging from informational notifications to warning alerts to critical interventions, with each alert including forensic metadata suitable for regulatory compliance and audit requirements. A comprehensive security architecture may combine TLS encryption, cryptographic hashing at the edge, and blockchain recording to provide an immutable audit trail.

In accordance with still further aspects of the invention, the platform incorporates a digital twin simulation environment for generating synthetic training data to train machine learning models. The digital twin models multiple physical phenomena including electromagnetic wave propagation, thermal effects, and fluid dynamics, enabling the generation of labeled synthetic datasets that cover a wide range of container types, liquid compositions, and contamination scenarios. A sim-to-real feedback loop continuously uses real-world sensor data to refine and calibrate the digital twin simulation, which in turn generates improved synthetic data for subsequent model training, creating a self-improving system. This approach addresses the challenge of obtaining large quantities of labeled training data in industrial settings.

In some aspects, the platform extends from passive monitoring to active control, as exemplified by a sump pump control application. A noninvasive sensor may be positioned externally to a sump basin to determine fluid level without physical contact with the fluid, eliminating the risk of mechanical failure from debris, corrosion, or wear associated with traditional float switches. An edge processor may execute a machine learning model that determines optimal pump activation state based on a predictive hysteresis model incorporating not only current fluid level but also rate of change and external environmental data such as weather forecasts. In some aspects, the system may operate in a dual-mode configuration with a traditional float switch for redundancy, wherein the noninvasive sensor acts as the primary controller while monitoring the state of the float switch to detect mechanical failures.

In accordance with yet further aspects of the invention, the platform integrates a blockchain trust layer to create an immutable audit trail for all sensor data and machine learning predictions. This blockchain-enabled ecosystem may support novel applications including parametric insurance products wherein smart contracts automatically execute insurance payouts based on blockchain-recorded sensor determinations, asset-backed financing against verified inventory through automated collateral valuation, and verifiable sustainability credits based on monitored environmental data. The integration of blockchain technology enables a single source of truth for all stakeholders including producers, logistics providers, insurers, and regulators.

The foregoing general description of the illustrative embodiments and the following detailed description thereof are merely exemplary aspects of the teachings of this disclosure and are not restrictive.

### BRIEF DESCRIPTION OF THE DRAWINGS

Non-limiting and non-exhaustive examples are described with reference to the following figures.

FIG. 1 illustrates a flowchart of a process for generating synthetic training data, according to aspects of the present disclosure.

FIG. 2 illustrates a flowchart of a process for a machine learning model lifecycle, according to aspects of the present disclosure.

FIG. 3 illustrates a conceptual diagram of a three-dimensional virtual simulation environment for a digital twin, according to aspects of the present disclosure.

FIGS. 4A-4C illustrate a blockchain-enabled ecosystem architecture diagram, according to aspects of the present disclosure.

FIG. 5 illustrates a multi-industry universal platform architecture, according to aspects of the present disclosure.

FIG. 6 illustrates an advanced deployment and infrastructure architecture, according to aspects of the present disclosure.

FIG. 7 illustrates a hardware block diagram of a modular sensor node, according to aspects of the present disclosure.

FIG. 8 illustrates a sump pump monitoring system, according to aspects of the present disclosure.

FIG. 9 illustrates a flowchart of a process for detecting contamination using dielectric fingerprinting, according to aspects of the present disclosure.

FIG. 10 illustrates a dual-use communication and sensing architecture using a wireless communication node, according to aspects of the present disclosure.

FIG. 11 illustrates a graph of a multi-frequency dielectric fingerprint comparison, according to aspects of the present disclosure.

FIG. 12 illustrates a layered data pipeline architecture for integrity monitoring, according to aspects of the present disclosure.

FIG. 13 illustrates a flowchart of a method for over-the-air machine learning model updates, according to aspects of the present disclosure.

FIGS. 14A and 14B illustrate a state diagram of a float sensor redundancy system, according to aspects of the present disclosure.

FIG. 15 illustrates a block diagram of an exemplary embodiment of a processing system, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

A detailed description of systems, devices, and methods consistent with embodiments of the present disclosure is provided below. While several embodiments are described, it should be understood that disclosure is not limited to any one embodiment, but instead encompasses numerous alternatives, modifications, and equivalents. In addition, while numerous specific details are set forth in the following description in order to provide a thorough understanding of the embodiments disclosed herein, some embodiments can be practiced without some or all of these details. Moreover, for the purpose of clarity, certain technical material that is known in the related art has not been described in detail in order to avoid unnecessarily obscuring the disclosure.

In accordance with at least one aspect of the present disclosure, a method for noninvasive monitoring of containerized assets across multiple industries is described. In some aspects, the method may include positioning a sensor externally to a container without penetrating a wall of the container. In some aspects, the method may further include acquiring sensor data from the sensor, the sensor data indicative of a property of contents within the container. In some aspects, the method may further include loading, onto an edge computing module, a machine learning model selected from a plurality of industry-specific machine learning models based on a target industry associated with the container. In some aspects, the method may further include processing the sensor data using the loaded machine learning model to generate a prediction regarding the contents of the container. In some aspects, the method may further include transmitting the prediction to a remote computing system.

In some aspects, the sensor may comprise a frequency-modulated continuous wave (FMCW) radar module configured to determine a fluid level within the container based on radar return signals propagated through the wall of the container.

In some aspects, the sensor may further comprise a dielectric sensor array configured to interrogate the contents of the container with electromagnetic signals across a plurality of frequencies to measure dielectric properties of the contents.

In some aspects, the method may further include constructing a dielectric spectrum based on the measured dielectric properties across the plurality of frequencies, wherein the dielectric spectrum serves as a fingerprint for the contents of the container. In some aspects, the method may further include comparing the dielectric spectrum to a baseline dielectric fingerprint to detect a deviation indicative of contamination, adulteration, or degradation of the contents. In some aspects, the method may further include classifying a contaminant type based on a shape and magnitude of the deviation using the loaded machine learning model.

In some aspects, the plurality of industry-specific machine learning models may comprise at least two of: a spirits aging model configured to predict at least one of fill level, alcohol proof, or maturation state; a fuel integrity model configured to predict at least one of fill level or contamination status; a pharmaceutical model configured to predict formulation integrity; a water quality model configured to predict contamination status; or a sump pump model configured to predict fluid level for pump control.

In some aspects, the method may further include generating a cryptographic hash of the sensor data at the edge computing module. In some aspects, the method may further include recording the cryptographic hash on a distributed blockchain ledger to create an immutable audit trail.

In some aspects, the method may further include receiving an updated machine learning model from the remote computing system via an over-the-air transmission. In some aspects, the method may further include replacing the loaded machine learning model with the updated machine learning model on the edge computing module.

In some aspects, the method may further include performing a calibration measurement to determine electromagnetic characteristics of the wall of the container. In some aspects, the method may further include subtracting an electromagnetic contribution of the wall from the sensor data to isolate a dielectric signature of the contents.

In some aspects, the container may comprise a wooden barrel and the calibration measurement may account for variations in moisture content of the wooden barrel.

In some aspects, the method may further include performing a first measurement using a first sensing modality to determine a physical property of the contents. In some aspects, the method may further include performing a second measurement using a second sensing modality to determine a compositional property of the contents. In some aspects, the method may further include comparing the first measurement and the second measurement to detect a volume-neutral integrity event wherein the physical property remains substantially unchanged while the compositional property has changed.

In accordance with at least one aspect of the present disclosure, a method for generating synthetic training data for a container monitoring system is described. In some aspects, the method may include defining, within a digital twin simulation environment, a virtual container having specified geometry and material properties. In some aspects, the method may further include configuring one or more virtual sensors within the digital twin simulation environment. In some aspects, the method may further include executing a physics simulation to model electromagnetic wave propagation through the virtual container and interaction with a virtual enclosed medium. In some aspects, the method may further include generating raw synthetic sensor data based on the physics simulation. In some aspects, the method may further include labeling the raw synthetic sensor data with ground truth parameters. In some aspects, the method may further include, based on the labeled raw synthetic sensor data, outputting a labeled training dataset for training a machine learning model.

In some aspects, the physics simulation may further model at least one of thermal effects or fluid dynamics within the virtual container.

In some aspects, configuring the one or more virtual sensors may include configuring at least one of a virtual FMCW radar sensor or a virtual dielectric sensor array.

In some aspects, the method may further include applying domain randomization by varying at least one of container wall thickness, liquid permittivity, ambient temperature, or sensor position within defined ranges across a plurality of synthetic data samples.

In some aspects, the ground truth parameters may include at least one of material composition, purity, fill level, contamination state, or maturation state.

In some aspects, the method may further include collecting real-world sensor data from a physical sensor monitoring a physical container. In some aspects, the method may further include identifying a domain gap between the raw synthetic sensor data and the real- world sensor data. In some aspects, the method may further include updating the digital twin simulation environment to reduce the domain gap.

In some aspects, updating the digital twin simulation environment may include adjusting at least one of a material property, a geometric parameter, or a noise model within the digital twin simulation environment.

In some aspects, the material properties of the virtual container may include electromagnetic properties including at least one of permittivity, conductivity, or thickness.

In some aspects, the method may further include training the machine learning model using the labeled training dataset. In some aspects, the method may further include deploying the trained machine learning model to an edge computing module of a physical sensor node. In some aspects, the method may further include processing real-world sensor data using the deployed machine learning model to generate predictions regarding contents of a physical container.

In some aspects, the method may further include identifying low-confidence predictions generated by the deployed machine learning model. In some aspects, the method may further include using the low-confidence predictions to refine parameters of the digital twin simulation environment for generating improved synthetic training data.

In accordance with at least one aspect of the present disclosure, a method for creating a tamper-proof audit trail for container monitoring data is described. In some aspects, the method may include acquiring sensor data from a noninvasive sensor monitoring a container. In some aspects, the method may further include generating a cryptographic hash of the sensor data at an edge computing device. In some aspects, the method may further include transmitting the sensor data and the cryptographic hash to a remote server over an encrypted communication channel. In some aspects, the method may further include processing the sensor data using a machine learning model to generate a prediction regarding contents of the container. In some aspects, the method may further include recording the cryptographic hash on a distributed blockchain ledger to create an immutable record of the sensor data.

In some aspects, the encrypted communication channel may comprise a Transport Layer Security (TLS) encrypted channel.

In some aspects, the method may further include generating an alert based on the prediction. In some aspects, the alert may include forensic metadata comprising the sensor data, the prediction, a confidence score, and a timestamp.

In some aspects, the alert may be classified into one of a plurality of severity tiers comprising an informational notification tier, a warning alert tier, and a critical intervention tier based on a magnitude of a detected deviation from an expected condition.

In some aspects, the method may further include automatically executing a smart contract deployed on the distributed blockchain ledger based on the recorded cryptographic hash. In some aspects, the smart contract may trigger a predefined action when a condition defined in the smart contract is met by the prediction.

In some aspects, the predefined action may comprise an automated insurance payout triggered by detection of a contamination event indicated by the prediction.

In some aspects, the method may further include generating a compliance report based on the prediction and the immutable record on the distributed blockchain ledger. In some aspects, the compliance report may be formatted for submission to a regulatory authority.

In some aspects, the sensor data may comprise at least one of a dielectric property measurement across a plurality of frequencies or a fluid level measurement derived from radar return signals.

In some aspects, the method may further include constructing a dielectric spectrum based on the dielectric property measurement across the plurality of frequencies. In some aspects, the method may further include comparing the dielectric spectrum to a baseline dielectric fingerprint. In some aspects, the method may further include detecting a contamination event based on a deviation between the dielectric spectrum and the baseline dielectric fingerprint exceeding a predefined threshold.

In some aspects, the method may further include analyzing a time-series of sensor data collected over a period of time to determine a rate of change of a measured property. In some aspects, the method may further include predicting a future contamination level or a time- to-failure based on the rate of change.

In certain embodiments, any one or more of the above aspects may be practiced using a non-transitory computer-readable medium that, when executed by a processor, causes the processor to practice a given aspect. In certain other embodiments, any one or more of the above aspects may be practiced using a system having a noninvasive sensor configured for external mounting to a container and a processor configured to practice a given aspect.

Industries that rely on containerized liquids face a technical problem rooted in the physical constraints of noninvasive sensing. When a sensor is positioned outside a container wall, electromagnetic signals must propagate through the wall material before interacting with the liquid inside. The wall introduces reflections, attenuation, and phase distortion that vary depending on wall material, thickness, and condition, and that are entangled with the electromagnetic response of the liquid itself. Extracting accurate information about the liquid from the corrupted return signal is a signal processing challenge that conventional single-modality sensing systems do not adequately solve, particularly when the goal is to characterize not merely the level of the liquid but also its composition and integrity. This problem is compounded by a second technical limitation: existing noninvasive monitoring systems are designed as fixed-function hardware optimized for a single container type and a single liquid, requiring separate hardware platforms for each application and preventing economies of scale. A third technical limitation arises in the machine learning domain: training accurate characterization models for each new container type, liquid, and contamination scenario conventionally requires collecting large quantities of labeled real-world sensor data, which is expensive, time-consuming, and in many cases destructive to the product being monitored. These three technical problems, taken together, have prevented the development of a unified noninvasive monitoring platform capable of serving multiple industries from a common hardware base.

The present disclosure addresses these technical problems through a combination of hardware and software improvements to the monitoring system itself. At the sensing layer, the disclosed system employs a multi-modal sensor fusion architecture that combines frequency-modulated continuous wave (FMCW) radar with multi-frequency dielectric fingerprinting to acquire two physically distinct types of measurement from the same container: radar return signals that are processed to determine fluid level, and dielectric property measurements across a range of frequencies that are processed to construct a permittivity spectrum serving as a compositional fingerprint of the liquid. The fusion of these two sensing modalities enables the system to detect integrity events that neither modality can detect alone, including volume-neutral events where the liquid level remains unchanged but the composition has been altered, such as dilution or substitution. A dynamic container-noise subtraction module isolates the dielectric signature of the liquid from the electromagnetic contribution of the container wall by subtracting the wall's measured or modeled electromagnetic characteristics from the composite return signal, improving the signal-to-noise ratio of the compositional measurement.

At the platform layer, a software-defined adaptation architecture decouples the hardware configuration from the monitoring application by providing a model adaptation layer that dynamically loads and executes different pre-trained machine learning models on a common edge computing module based on the target industry and container type. This architectural separation enables a single standardized hardware platform to be reconfigured for different applications through software rather than hardware redesign. To address the training data bottleneck, the system incorporates a digital twin simulation environment that generates labeled synthetic sensor data by modeling multi-physics interactions, including electromagnetic wave propagation, thermal effects, and fluid dynamics, within a virtual representation of the container and its contents. A feedback mechanism uses low-confidence predictions from deployed models to identify discrepancies between the synthetic and real- world data distributions and refines the simulation parameters accordingly, progressively improving the fidelity of the synthetic data and the accuracy of subsequently trained models without requiring additional real-world data collection.

These technical improvements produce measurable benefits in the functioning of the monitoring system. The multi-modal sensor fusion architecture improves the system's detection capability by enabling identification of integrity events that are invisible to single- modality sensors, such as detecting that a spirit has been diluted with water even though the fluid level in the barrel has not changed. The container-noise subtraction module improves measurement accuracy by removing a physical source of signal corruption, allowing the dielectric fingerprint of the liquid to be characterized with greater precision across varying container materials and conditions, including wooden barrels with non-uniform moisture content. The software-defined adaptation layer reduces the computational and logistical burden of multi-industry deployment by eliminating the need for separate hardware designs for each application, and enables over-the-air deployment of new monitoring capabilities to installed sensor nodes without physical hardware modification.

The digital twin simulation environment and feedback mechanism reduce the time, cost, and data requirements associated with training new machine learning models for previously unseen container types or liquid formulations by generating synthetic training datasets that cover a wide range of physical conditions through parametric variation within the simulation. The continuous feedback between deployed models and the simulation creates a self-improving system in which model accuracy increases over time without proportional increases in real-world data collection effort. At the data integrity layer, cryptographic hashing of sensor data at the edge computing module at the point of data acquisition, combined with recording of the hash on a distributed ledger, creates a verifiable chain of data provenance from the physical sensor measurement through processing and storage, providing a technical mechanism for detecting any post-acquisition modification of the sensor data.

Industries that store, transport, or process liquids in containers face numerous challenges related to monitoring the state, integrity, and composition of containerized contents. Traditional invasive sensing techniques require physical penetration of the container wall or direct contact with the liquid, which compromises container integrity by introducing potential leak points and creating contamination pathways that can affect product quality or safety. Furthermore, physical penetration is often not feasible for sealed containers such as aging barrels or sterile pharmaceutical vessels. Mechanical sensors, including float switches and other electromechanical devices, are prone to failure due to debris accumulation, corrosion, wear, and mechanical fatigue, making such sensors unreliable for applications where failure can result in catastrophic consequences such as flooding or contamination events.

Manual sampling and laboratory analysis represent another conventional approach to liquid characterization and contamination detection. However, manual sampling is expensive, time-consuming, and provides only a point-in-time snapshot rather than continuous monitoring. Manual sampling also introduces the risk of contamination during the sampling process itself and is not scalable for large inventories or distributed assets. Existing noninvasive sensing systems are typically purpose-built for single applications, resulting in fragmented solutions that cannot be adapted to different industries, container types, or liquid formulations without substantial hardware redesign. This fragmentation increases manufacturing costs, complicates inventory and logistics, and limits the ability to leverage economies of scale.

Deploying machine learning models for liquid characterization and integrity monitoring presents additional challenges related to obtaining large, labeled training datasets. Collecting real-world data by manually sampling containers under every possible condition is prohibitively expensive, time-consuming, and in some cases destructive. For applications involving aging processes, data collection can require years to complete. Furthermore, existing systems lack robust mechanisms for ensuring data integrity, auditability, and regulatory compliance. Centralized databases are inherently alterable by privileged administrators and do not provide the level of trust and verifiability used in multi-party transactions, regulatory submissions, or insurance claims. These limitations create barriers to the development of novel business models such as parametric insurance, asset-backed financing, and verifiable sustainability credits that depend on trusted, unalterable data sources.

The present disclosure relates to a universal, noninvasive integrity monitoring and control platform that may address the foregoing challenges and more through a modular, externally-positioned sensor system combined with a software-defined architecture. The platform may comprise a standardized hardware configuration that can be adapted to serve multiple disparate industries (including, but not limited to, spirits aging, military fuel logistics, pharmaceutical manufacturing, municipal water treatment, and residential sump pump control) by loading different pre-trained machine learning models onto the same hardware. This software-defined approach decouples the hardware from the application, enabling a single hardware platform to be reconfigured for different container types, liquid formulations, or industry-specific requirements without hardware redesign, thereby reducing manufacturing costs through economies of scale and simplifying inventory, logistics, and support operations.

The platform may employ a multi-modal sensor fusion approach that integrates multiple sensing modalities to provide comprehensive analysis of containerized contents. In certain embodiments, the sensor system combines FMCW radar for precise, noninvasive fluid level detection with dielectric fingerprinting for content characterization and contamination detection. By sweeping an electromagnetic signal across a range of frequencies and measuring the liquid's complex permittivity at each frequency, the system may construct a dielectric spectrum that serves as a unique fingerprint for the liquid, enabling detection of contamination, adulteration, or degradation without physical contact with the liquid or penetration of the container wall. The platform may extend beyond passive monitoring to active control applications, such as intelligent sump pump control that incorporates predictive hysteresis based on current fluid levels, rate of change, and external data sources including weather forecasts, thereby replacing unreliable mechanical float switches with a noninvasive, failure- resistant control system.

To address the challenge of obtaining large, labeled training datasets for machine learning model development, the platform may incorporate a digital twin simulation environment that generates synthetic training data by modeling multi-physics interactions including electromagnetic wave propagation, thermal effects, and fluid dynamics within a virtual representation of the container and its contents. A continuous feedback loop between real-world sensor data and the digital twin simulation may refine simulation parameters over time, improving the fidelity of synthetic data and the accuracy of trained models. To address data integrity, auditability, and regulatory compliance requirements, the platform may incorporate blockchain technology to create an immutable audit trail by cryptographically hashing sensor data at the point of collection and recording the hash on a distributed ledger. This blockchain-enabled trust layer may serve as a foundation for novel business models including parametric insurance products, asset-backed financing arrangements, and verifiable sustainability credits that depend on trusted, unalterable data sources accessible to multiple stakeholders including producers, logistics providers, insurers, regulators, and financial institutions.

The software-defined architecture described herein may provide several technical advantages over conventional purpose-built sensing systems. In certain embodiments, the universal hardware platform enables a single hardware configuration to serve multiple disparate industries through the loading of different pre-trained machine learning models, which may reduce manufacturing costs through economies of scale, simplify inventory management and logistics operations, and enable rapid deployment to new applications without hardware redesign. The software-defined nature of the platform may allow new capabilities to be deployed to existing sensor nodes via over-the-air updates, providing a future-proof solution that can adapt to new container types, liquid formulations, or industry-specific requirements as they emerge.

The multi-modal sensor fusion approach may provide technical advantages over single-modality sensing systems by enabling comprehensive analysis that combines physical property measurements with compositional characterization. In certain embodiments, the combination of FMCW radar for fluid level detection with dielectric fingerprinting for content characterization enables detection of volume-neutral integrity events (such as dilution of a spirit with water) where the physical volume remains substantially unchanged but the compositional properties have changed. The multi-frequency dielectric spectrum construction may provide a rich data signature that enables not only detection of deviations from a baseline but also classification of specific contaminant types based on the unique shape and magnitude of spectral shifts, thereby providing actionable information for remediation rather than merely indicating that an anomaly has occurred.

The digital twin simulation environment and blockchain-enabled trust layer may provide technical advantages that extend beyond the sensing capabilities of the platform. In certain embodiments, the synthetic data generation pipeline addresses the data bottleneck that conventionally limits deployment of machine learning models for industrial sensing applications by enabling rapid development and training of highly accurate models without the expense, time, and potential destructiveness of collecting large real-world datasets. The continuous feedback loop between real-world sensor data and the digital twin simulation may create a self-improving system where models become progressively more accurate over time. The blockchain-enabled trust layer may provide an immutable audit trail that eliminates disputes regarding data integrity and provides a single source of truth accessible to multiple stakeholders, which may reduce fraud, compliance costs, and administrative overhead while enabling novel business models (including one or more of parametric insurance products, asset backed financing arrangements, or verifiable sustainability credits) that depend on trusted, unalterable data sources.

In the below-discussed figures, elements may be added, removed, modified, or rearranged without departing from the scope of the present disclosure.

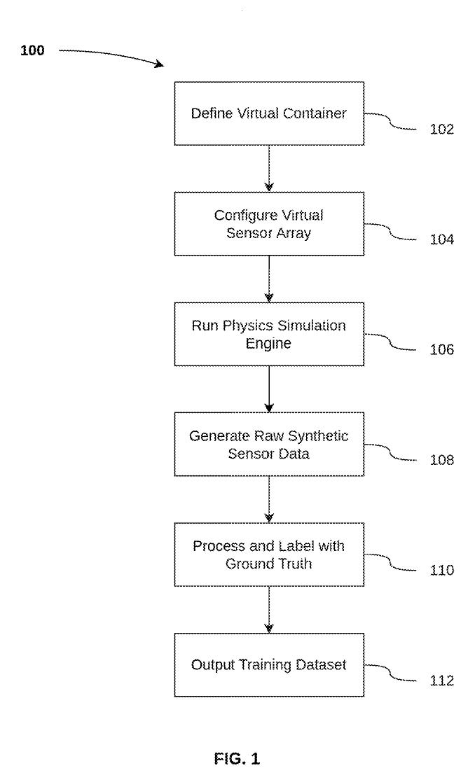

Referring to FIG. 1, a process 100 for generating synthetic training data is illustrated as a flowchart depicting a six-step pipeline that produces labeled datasets for machine learning model training. The process 100 may enable rapid development of training data without the expense, time, and potential destructiveness associated with collecting large real-world datasets through manual sampling of containers under varying conditions.

The process 100 begins with a step 102 of defining a virtual container within the digital twin simulation environment. In step 102, the physical attributes of the container may be specified, including the geometry of the container such as shape, dimensions, and/or wall curvature. Step 102 may further include specification of the material composition of the container walls, which may include any suitable one or more materials, such as wood for aging barrels, metal for fuel tanks, glass for pharmaceutical vials, plastic for sump basins, or composite materials. The content properties of the liquid or solid enclosed within the container may also be defined in step 102, including properties such as density, viscosity, and initial chemical composition. In certain embodiments, step 102 includes specification of the nature of the enclosed medium, such as whether the medium is a single-phase liquid, a multi-phase mixture, or a liquid with suspended particulates.

With continued reference to FIG. 1, the process 100 proceeds to a step 104 of configuring a virtual sensor array within the digital twin simulation environment. In step 104, a simulated array of one or more sensors may be set up to correspond to the sensor configuration that will be deployed on physical containers. The virtual sensor array configured in step 104 may include proximal sensors such as FMCW radar antennas and electromagnetic antennas positioned outside the container wall. Step 104 may further include configuration of embedded sensors such as piezoelectric elements, strain gauges, and/or embedded radio frequency antennas that are modeled as being positioned within or on the container structure. Environmental sensors for monitoring ambient conditions including any one or more of temperature, humidity, pressure, or vibration may also be configured in step 104. The configuration of the virtual sensor array in step 104 may define any one or more of the position, orientation, or operating parameters of each sensor type to match the intended physical deployment configuration.

The process 100 continues to a step 106 of running a physics simulation engine to model complex physical phenomena within the virtual container environment. In step 106, the simulation engine may model electromagnetic wave propagation through the container walls and the enclosed medium, including any one or more of reflections at material interfaces, dielectric interactions between the electromagnetic waves and the liquid, or multi-path effects caused by internal reflections within the container. The digital twin simulation environment executing step 106 may model multiple physical phenomena including any one or more of electromagnetic wave propagation, thermal effects (e.g., heat transfer and/or temperature gradients), and fluid dynamics including stratification, sedimentation, and convective mixing. Step 106 may model the interaction between thermal effects and electromagnetic properties, such as temperature-dependent changes in the dielectric constant of the liquid. The physics simulation engine used in step 106 may comprise any suitable simulation software such as Unity for real-time rendering or COMSOL for finite element analysis of electromagnetic and thermal phenomena.

As further shown in FIG. 1, a step 108 generates raw synthetic sensor data based on the simulated returns from all configured virtual sensors. The raw synthetic sensor data produced in step 108 may include any one or more of simulated radar return signals, dielectric property measurements across multiple frequencies, temperature readings, humidity measurements, or other sensor outputs corresponding to the virtual sensor array configured in step 104. The synthetic data generation pipeline may use domain randomization in step 108 by randomly varying parameters including any one or more of container wall thickness, liquid permittivity, ambient temperature, or sensor position within defined ranges to create diverse training datasets. In certain embodiments, the domain randomization applied in step 108 varies multiple parameters simultaneously across their defined ranges for each synthetic data sample, thereby producing a dataset that covers a wide range of potential real-world conditions and improves the robustness of trained models to variations in deployment environments.

The process 100 proceeds to a step 110 of processing and labeling the raw synthetic data with ground truth parameters. In step 110, the raw synthetic sensor data generated in step 108 may be automatically processed and labeled with the precise simulation parameters that were used to generate the data, including the material composition of the container and contents, the purity or contamination state of the liquid, the fill level within the container, and/or the maturation state for aging applications.

The process 100 concludes with a step 112 of outputting the labeled training dataset. The labeled synthetic data output in step 112 may be formatted for use in training machine learning models, including deep neural network architectures such as long short-term memory networks, convolutional neural networks, or transformer architectures. The training dataset output in step 112 may include paired input-output samples where the input comprises the synthetic sensor data and the output comprises the ground truth labels, enabling supervised learning approaches for training models to predict container content properties from sensor measurements.

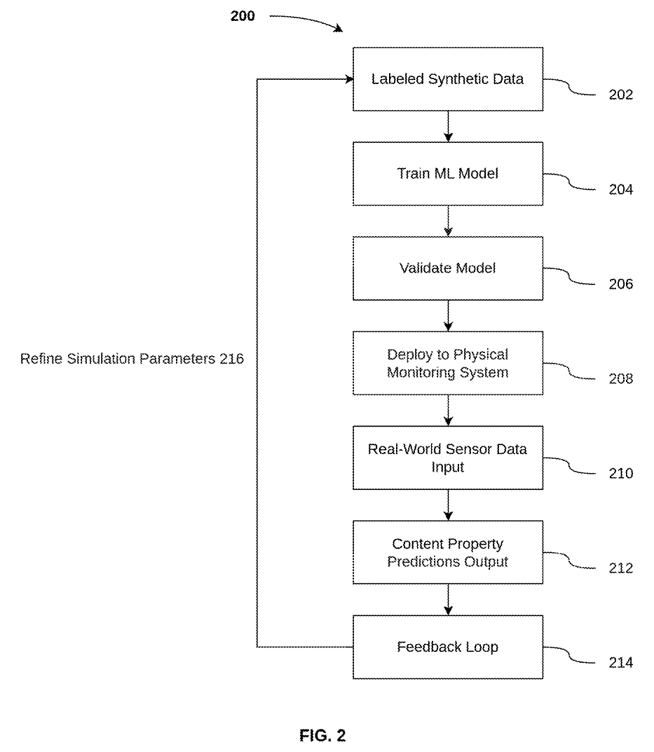

Referring to FIG. 2, a process 200 for the machine learning model lifecycle is illustrated as a flowchart depicting a multi-step pipeline that transforms labeled synthetic data into deployed predictive models and incorporates continuous improvement through feedback mechanisms. The process 200 may enable the development, validation, deployment, and ongoing refinement of machine learning models for noninvasive container monitoring applications.

The process 200 begins with a step 202 of receiving labeled synthetic data from the synthetic data generation pipeline described with reference to FIG. 1. In step 202, the labeled training dataset output from step 112 of the process 100 may be ingested for use in model training. The labeled synthetic data received in step 202 may include paired input-output samples where the input comprises synthetic sensor data including one or more of radar return signals, dielectric property measurements, or environmental sensor readings, and the output comprises ground truth labels including one or more of fill level, composition, purity, or contamination state.

With continued reference to FIG. 2, the process 200 proceeds to a step 204 of training a machine learning model using the labeled synthetic data received in step 202. In step 204, a machine learning model may be trained using deep neural network architectures including one or more of long short-term memory (LSTM) networks for temporal sequence modeling, convolutional neural networks (CNN) for spatial feature extraction, or transformer architectures for attention-based learning. The model architecture selected in step 204 may depend on the characteristics of the sensor data and the prediction task, with LSTM networks being suitable for time-series analysis of sensor readings, CNNs being suitable for extracting spatial patterns from multi-frequency dielectric spectra, and transformer architectures being suitable for capturing long-range dependencies in sequential data.

In certain embodiments, the machine learning model training performed in step 204 utilizes a federated learning approach where a global machine learning model is distributed to a plurality of distributed sensor nodes, each node monitoring a container. Local versions of the global model may be trained at each sensor node using local sensor data collected by that node. Updates from the local model versions may be aggregated to update the global machine learning model without transmitting the raw local sensor data to a central server. The federated learning approach may preserve data privacy by keeping raw sensor data at the edge while still enabling collaborative model improvement across a distributed sensor network. In certain embodiments, the federated learning approach enables training of models across multiple geographically distinct storage facilities without centralizing sensitive operational data.

In certain embodiments, the synthetic data generation used in step 202 and step 204 employs a generative adversarial network (GAN) trained on a dataset of real sensor data from a container monitoring system. The trained GAN may generate new synthetic sensor data that is statistically similar to the real sensor data, augmenting the physics-based synthetic data generated by the digital twin simulation. In certain embodiments, the GAN comprises a conditional GAN (cGAN) that is conditioned on a contaminant type, thereby generating synthetic fingerprints for specific rare contamination events that may be underrepresented in real-world datasets. The GAN-generated synthetic data may supplement the physics-based synthetic data to improve model robustness for edge cases and rare events.

As further shown in FIG. 2, the process 200 continues to a step 206 of validating the trained machine learning model. In step 206, the model trained in step 204 may undergo rigorous testing using one or more of cross-validation techniques, held-out test sets, or simulation-to-real gap analysis. The validation performed in step 206 may assess the model's ability to generalize from synthetic training data to real-world sensor data by comparing model predictions on synthetic test data with predictions on real-world sensor data collected from physical deployments. The simulation-to-real gap analysis performed in step 206 may identify discrepancies between the feature distributions of synthetic data and real-world data, which may inform subsequent refinement of the digital twin simulation parameters.

The process 200 proceeds to a step 208 of deploying the validated model to a physical monitoring system. In step 208, the machine learning model that has been validated in step 206 may be installed on an edge computing module of a sensor node equipped with a physical multi-modal sensor array including one or more of proximal sensors, embedded sensors, or environmental sensors. The deployment performed in step 208 may include model compression or quantization techniques to reduce the memory footprint and computational requirements of the model for execution on resource-constrained edge devices.

With continued reference to FIG. 2, a step 210 receives real-world sensor data input from live readings collected by the physical monitoring system. In step 210, the deployed sensor node may collect sensor data from the multi-modal sensor array during normal monitoring operations, including one or more of FMCW radar return signals, dielectric property measurements across multiple frequencies, or environmental sensor readings such as temperature and humidity.

A step 212 produces content property predictions output based on the real-world sensor data received in step 210. In step 212, the deployed machine learning model may process the real-world sensor data and generate predictions regarding the container contents, including one or more of composition, purity, fill level, maturation state, density, or viscosity. The predictions generated in step 212 may be transmitted to a cloud analytics platform for aggregation with predictions from other sensor nodes in a distributed monitoring network.

A step 214 implements a feedback loop that identifies low-confidence predictions and edge cases from the predictions generated in step 212. In step 214, predictions where the model confidence score falls below a threshold or where the sensor data exhibits characteristics not well-represented in the training data may be flagged for further analysis. The feedback loop implemented in step 214 may identify systematic discrepancies between model predictions and actual outcomes, which may indicate areas where the digital twin simulation does not accurately represent real-world conditions.

A step 216 refines simulation parameters by feeding improvements back to the synthetic data generation pipeline of the process 100. In step 216, the low-confidence predictions and edge cases identified in step 214 may be used to calibrate and improve the fidelity of the digital twin simulation. The sim-to-real domain adaptation performed in step 216 may identify a domain gap between synthetic data and real-world data by analyzing the feature distributions of both data types. The digital twin simulation may be updated by adjusting one or more of material properties, geometric parameters, or noise models to reduce the identified domain gap. The refined simulation parameters may be used to generate improved synthetic training data, which may be fed back to step 202 to train updated models with improved accuracy and robustness.

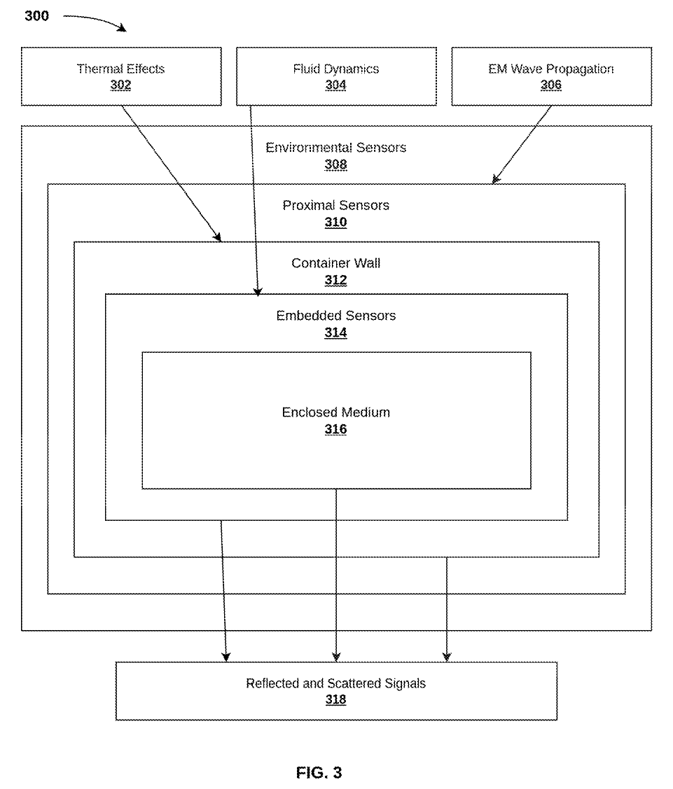

Referring to FIG. 3, a 3D virtual simulation environment 300 for the digital twin is illustrated as a conceptual diagram depicting the multi-physics modeling architecture used to generate synthetic sensor data. The 3D virtual simulation environment 300 may provide a comprehensive virtual representation of a container and the container's contents that enables simulation of complex physical interactions between electromagnetic signals, thermal phenomena, and/or fluid behavior.

The 3D virtual simulation environment 300 models three physics phenomena that affect sensor measurements and container content characterization. A thermal effects 302 phenomenon encompasses heat transfer mechanisms and temperature gradients within and around the container, which may affect one or more of the dielectric properties of the enclosed liquid, the material properties of the container wall, or the behavior of temperature-sensitive sensors. A fluid dynamics 304 phenomenon encompasses fluid behavior within the container, which may include one or more of stratification due to density differences, sedimentation of suspended particulates, or convective mixing caused by temperature gradients. An EM wave propagation 306 phenomenon encompasses the behavior of electromagnetic waves as the electromagnetic waves travel through the container structure and interact with the enclosed medium, which may include one or more of transmission through material interfaces, reflection at boundaries between materials with different dielectric properties, or absorption by the liquid or container materials.

With continued reference to FIG. 3, the 3D virtual simulation environment 300 depicts a series of concentric nested layers representing the physical structure of the monitored container and the sensor positions relative to that structure. An environmental sensors 308 layer forms the outermost layer of the nested structure and represents sensors positioned in the ambient environment surrounding the container. The environmental sensors 308 may monitor ambient conditions that affect sensor measurements and container content properties, including one or more of temperature, humidity, pressure, or vibration. The environmental data collected by the environmental sensors 308 may be used to compensate for environmental effects on sensor readings and to correlate environmental conditions with changes in container content properties.

A proximal sensors 310 layer is positioned inside the environmental sensors 308 layer and represents sensors positioned outside but in close proximity to the container wall. The proximal sensors 310 may include one or more of FMCW radar antennas for fluid level detection or electromagnetic antennas for dielectric property measurement. The proximal sensors 310 may perform noninvasive measurements by transmitting electromagnetic signals through the container wall and analyzing the returned signals without physical contact with the container contents.

A container wall 312 layer separates the external sensor layers from the internal container structure and represents the physical barrier between the proximal sensors 310 and the enclosed contents. The container wall 312 may comprise any one or more suitable materials, including, for example, any one or more of wood for aging barrels, metal for fuel tanks, glass for pharmaceutical vials, plastic for sump basins, or composite materials. The 3D virtual simulation environment 300 may model the electromagnetic properties of the container wall 312, including one or more of permittivity, conductivity, or thickness, which affect the transmission, reflection, and attenuation of electromagnetic signals passing through the container wall 312.

As further shown in FIG. 3, an embedded sensors 314 layer is positioned inside the container wall 312 and represents sensors that are physically integrated within or on the container structure. The embedded sensors 314 may include one or more of piezoelectric elements for vibration sensing, strain gauges for mechanical stress measurement, or embedded radio frequency antennas for internal electromagnetic measurements. In certain embodiments, the embedded sensors 314 provide supplementary data that complements the noninvasive measurements from the proximal sensors 310.

An enclosed medium 316 forms the innermost layer of the nested structure and represents the fluid or solid contents being monitored within the container. The enclosed medium 316 may comprise one or more of a single-phase liquid, a multi-phase mixture, or a liquid with suspended particulates. The 3D virtual simulation environment 300 may model the electromagnetic properties of the enclosed medium 316, including one or more of complex permittivity, conductivity, or frequency-dependent dielectric response, which determine how electromagnetic signals interact with the enclosed medium 316.

With continued reference to FIG. 3, a reflected and scattered signals 318 block captures the simulation outputs generated by the 3D virtual simulation environment 300. The reflected and scattered signals 318 may include one or more of scattering data representing the dispersion of electromagnetic energy by inhomogeneities in the enclosed medium 316 or container wall 312, attenuation data representing the reduction in signal strength as electromagnetic waves propagate through materials, phase shift data representing changes in the phase of electromagnetic signals caused by propagation through materials with different dielectric properties, multi-path reflection data representing signals that have undergone multiple reflections within the container structure, or dielectric response data representing the frequency-dependent permittivity of the enclosed medium 316. The reflected and scattered signals 318 may be processed to generate the raw synthetic sensor data described with reference to step 108 of the process 100.

In certain embodiments, the 3D virtual simulation environment 300 supports optimization of sensor placement using reinforcement learning techniques. A reinforcement learning agent may explore a state space representing possible configurations of a plurality of sensors around the container modeled in the 3D virtual simulation environment 300. The reinforcement learning agent may simulate sensor readings for different sensor configurations within the 3D virtual simulation environment 300 and evaluate each configuration based on a reward function. The reward function may be designed to maximize the prediction accuracy of a machine learning model trained on data from the simulated sensor configuration. In certain embodiments, the reward function may be designed to maximize the accuracy of detecting a small leak by optimizing the placement of antennas to minimize multi-path interference. The reinforcement learning agent may identify an optimal sensor configuration based on the reward function, which may then be used to guide physical sensor deployment on real containers.

In some aspects, the platform architecture may support alternative approaches to edge-cloud partitioning beyond the distributed inference model described herein. In one alternative configuration, a cloud-only inference architecture may transmit raw sensor data from the sensor node to the Cloud Analytics Platform 528 for centralized processing, where full-precision machine learning models execute on cloud computing resources without requiring compressed models at the edge. The cloud-only configuration may be suitable for deployment environments with reliable, high-bandwidth connectivity where latency requirements are less stringent, such as warehouse-scale monitoring of stationary containers. In another alternative configuration, a tiered inference architecture may perform preliminary anomaly screening at the edge using a lightweight classifier and transmit sensor data to the cloud for detailed characterization using a higher-capacity model when the lightweight classifier detects a potential anomaly exceeding a screening threshold. In some aspects, the selection between edge-only, cloud-only, and tiered inference architectures may be performed dynamically by the Software-Defined Adaptation Layer 506 based on current network connectivity status, available edge computing resources, and the criticality of the monitoring application.

In some aspects, alternative approaches to data integrity verification may be used in place of or in combination with the blockchain-based audit trail. In one alternative configuration, a public key infrastructure (PKI) based verification approach may use a chain of digitally signed certificates to establish data provenance from the sensor node through edge processing to cloud storage, without requiring a distributed blockchain ledger. In another alternative configuration, a trusted platform module (TPM) integrated within the Edge Computing Module 524 may perform hardware-attested data integrity verification, generating tamper-evident attestation records that can be independently validated without blockchain transactions. In some aspects, the platform may support configurable data integrity verification where the operator selects among blockchain-based, PKI-based, TPM-based, or hybrid verification approaches based on the security requirements and infrastructure constraints of the deployment environment.

In some aspects, the Dynamic Container-Noise Subtraction Module 538 may employ alternative approaches to isolating the dielectric signature of the liquid from the electromagnetic contribution of the container wall. In one alternative configuration, an analytical subtraction approach may use a physics-based model of electromagnetic wave propagation through the container wall material, parameterized by the measured or known material properties and wall thickness, to calculate and subtract the wall contribution without requiring a machine learning model. In another alternative configuration, a reference measurement approach may use a reference sensor element positioned adjacent to an empty portion of the container wall to directly measure the wall contribution and subtract the measured reference from the sensing measurement. The reference measurement approach may be particularly suitable for containers where wall properties vary significantly across the surface, such as wooden barrels with non-uniform moisture distribution.

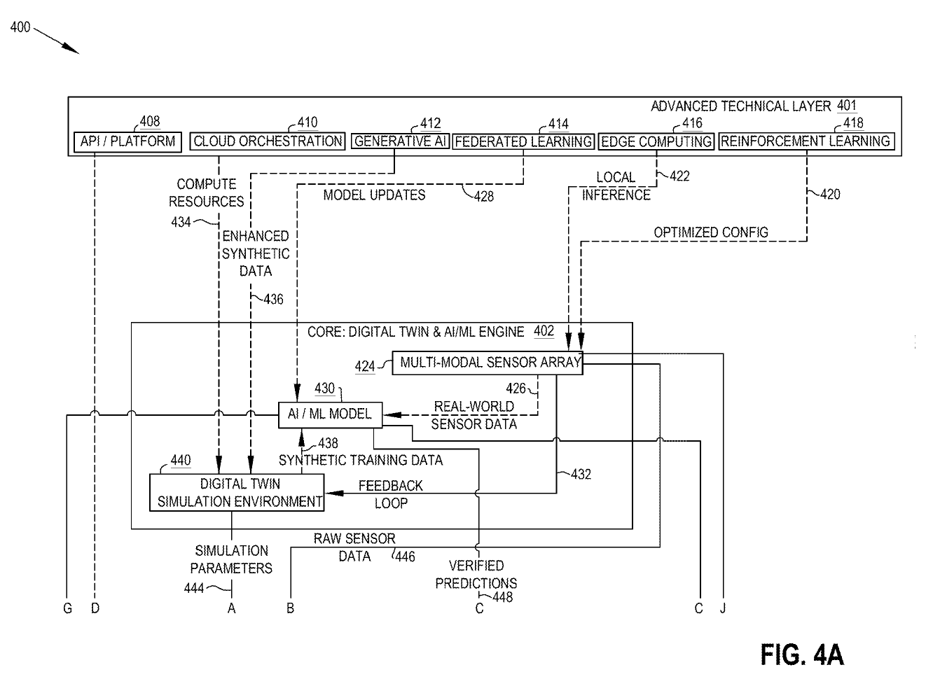

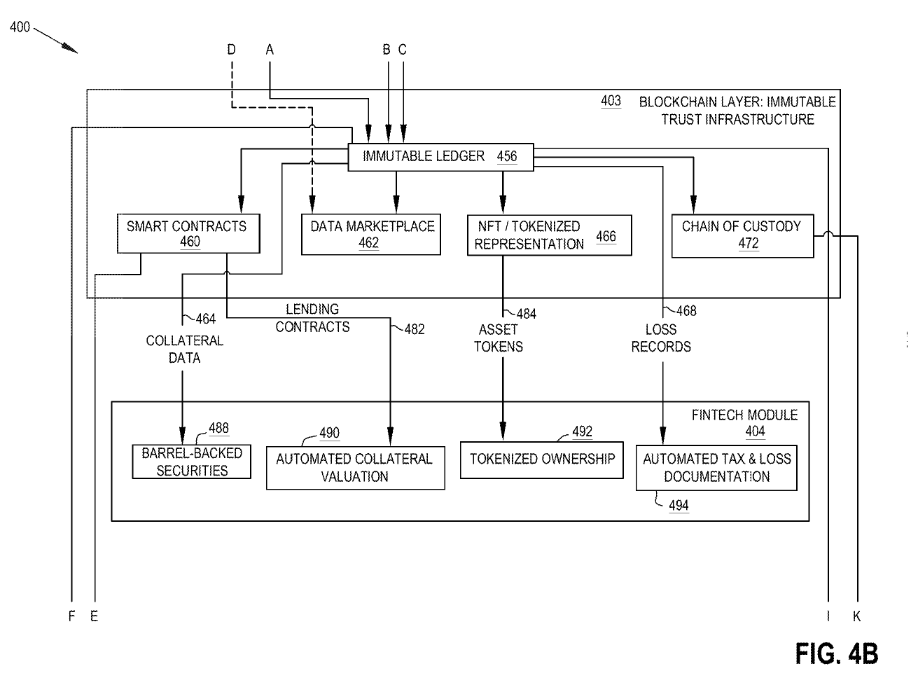

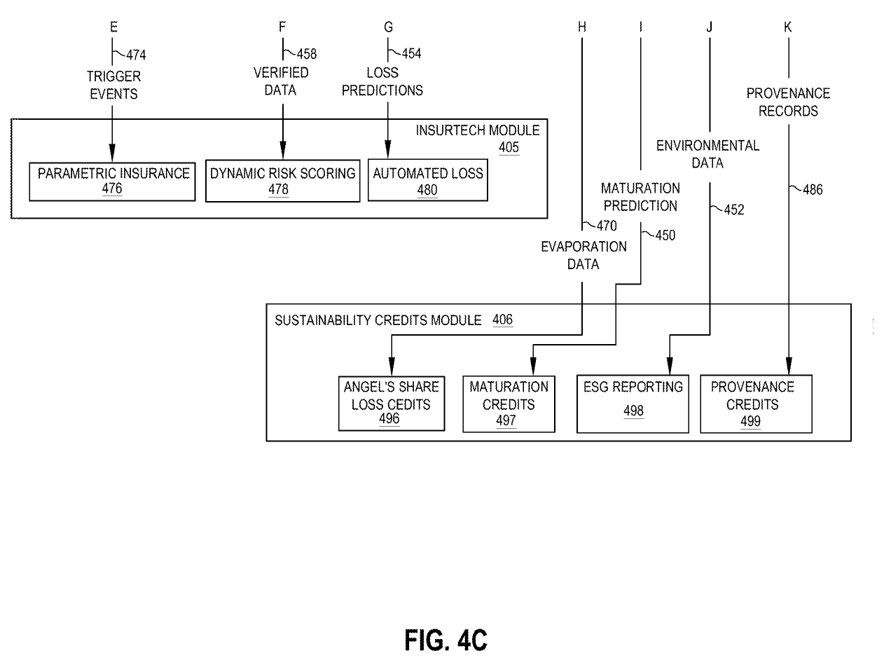

Referring to FIGS. 4A-4C, a Blockchain-Enabled Ecosystem Architecture 400 is illustrated as a comprehensive architecture diagram organized into four distinct layers that integrate sensing, machine learning, and distributed ledger technologies to provide a trusted data infrastructure for container monitoring applications. The Blockchain-Enabled Ecosystem Architecture 400 may enable the creation of novel business models including one or more of parametric insurance products, asset-backed financing arrangements, or verifiable sustainability credits that depend on trusted, unalterable data sources accessible to multiple stakeholders.

An Advanced Technical Layer 401 forms the uppermost layer of the Blockchain- Enabled Ecosystem Architecture 400 and contains modules that provide advanced computational capabilities for the platform. The Advanced Technical Layer 401 may include an API 408 that provides application programming interface access for third-party integration and platform connectivity. A Cloud Orchestration 410 module within the Advanced Technical Layer 401 may provide scalable cloud computing resources for running large-scale simulations and model training operations. A Generative AI 412 module may provide enhanced data synthesis capabilities using generative adversarial networks or other generative model architectures to augment physics-based synthetic data generation. A Federated Learning 414 module may enable distributed model training across multiple sensor nodes without centralizing raw sensor data, thereby preserving data privacy while enabling collaborative model improvement.

With continued reference to FIGS. 4A-4C, the Advanced Technical Layer 401 further includes an Edge Computing 416 module that enables on-device inference at sensor nodes, reducing latency and bandwidth requirements by performing predictions locally rather than transmitting raw sensor data to cloud servers. A Reinforcement Learning 418 module may provide sensor placement optimization capabilities as described with reference to the 3D virtual simulation environment 300. The Reinforcement Learning 418 module may generate an Optimized Config 420 output representing an optimal sensor configuration determined through reinforcement learning exploration of the sensor placement state space. The Edge Computing 416 module may perform Local Inference 422 using any one or more machine learning models deployed to edge devices, enabling real-time predictions without cloud connectivity.

A Core Layer 402 forms the central processing layer of the Blockchain-Enabled Ecosystem Architecture 400 and comprises the sensing, machine learning, and simulation components that generate and process container monitoring data. The Core Layer 402 may include a Multi-Modal Sensor Array 424 that collects sensor data from multiple sensing modalities including one or more of FMCW radar, dielectric sensors, or environmental sensors. The Multi-Modal Sensor Array 424 may generate Real-World Sensor Data 426 comprising live sensor readings collected from physical container monitoring deployments. A Feedback Loop 432 connects the Multi-Modal Sensor Array 424 to the Digital Twin Simulation Environment 440.

As further shown in FIGS. 4A-4C, the Core Layer 402 includes an AI/ML Model Training and Inference 430 component that performs machine learning model training using synthetic and real-world data and executes trained models to generate predictions. The AI/ML Model Training and Inference 430 component may receive Model Updates 428 from the Federated Learning 414 module.

The Core Layer 402 may receive Compute Resources 434 that provide processing capacity for model training and simulation operations. The Compute Resources 434 may be allocated dynamically based on computational demands using the Cloud Orchestration 410 module of the Advanced Technical Layer 401. The Core Layer 402 may receive Enhanced Synthetic Data 436 generated using the Generative AI 412 module to augment physics-based synthetic data with GAN-generated samples that capture rare events and edge cases. Synthetic Training Data 438 may be generated by a Digital Twin Simulation Environment 440 that models multi-physics interactions within virtual container representations as described with reference to the 3D virtual simulation environment 300.

With continued reference to FIGS. 4A-4C, the Digital Twin Simulation Environment 440 may generate Simulation Parameters 444 that define the virtual container configuration including one or more of geometry, material properties, or content characteristics, which may be integrated with Immutable Ledger 456. API 408 may perform third-party integration to integrate the system with a Digital Marketplace 462. The Multi- Modal Sensor Array 424 may generate Raw Sensor Data 446 that may be received by Immutable Ledger 456.

The Core Layer 402 may generate multiple types of prediction outputs based on the application domain and deployed machine learning model. Verified Predictions 448 may comprise predictions that have been validated against ground truth data or cross-validated using multiple sensing modalities, generated via AI/ML Model Training and Inference 430 and received by Immutable Ledger 456. Maturation Predictions 450 may comprise predictions regarding the aging state of products such as spirits in barrels, including one or more of chemical composition changes, flavor profile development, or optimal bottling time. Maturation Predictions 450 may be used in the generation of Maturation Credits 497. Environmental Data 452 may comprise processed environmental sensor readings including one or more of temperature, humidity, or pressure that affect container content properties. Environmental Data 452 may be used for ESG Reporting 498. Loss Predictions 454 may comprise predictions regarding product loss due to one or more of evaporation, leakage, or theft, which may be used for inventory management and insurance applications. Loss Predictions 454 may be used for Automated Loss Quantification 480.

With continued reference to FIGS. 4A-4C, a Blockchain Layer 403 forms the trust infrastructure layer of the Blockchain-Enabled Ecosystem Architecture 400 and provides immutable data recording and automated execution capabilities that enable multi-party transactions and regulatory compliance. The Blockchain Layer 403 may include an Immutable Ledger 456 that stores cryptographic hashes of sensor data and predictions in a distributed, tamper-proof manner. The Immutable Ledger 456 may record transactions across a network of distributed nodes such that no single party can alter historical records without detection, thereby providing a trusted data foundation accessible to multiple stakeholders including one or more of producers, logistics providers, insurers, regulators, or financial institutions.

Immutable Ledger 456 may include Verified Data 458 comprising sensor measurements and predictions that have been cryptographically signed at the point of collection and recorded on the Immutable Ledger 456. The Verified Data 458 may provide a single source of truth for container state information that can be independently verified by any authorized party. Verified Data 458 may be used for Dynamic Risk Scoring 478. Smart Contracts 460 within the Blockchain Layer 403 may provide automated execution capabilities that trigger predefined actions when specified conditions are met. The Smart Contracts 460 may execute without human intervention based on data recorded on the Immutable Ledger 456, enabling automated business processes including one or more of insurance claim processing, collateral valuation updates, or regulatory reporting.

The Blockchain Layer 403 may further include a Data Marketplace 462 that enables authorized parties to access and transact using verified sensor data and predictions. The Data Marketplace 462 may facilitate data sharing between stakeholders while maintaining data provenance and access controls. Collateral Data 464 within the Blockchain Layer 403 may comprise verified asset state information used for financial applications including one or more of lending, insurance underwriting, or securities issuance. Collateral Data 464 may be used in the creation and/or maintenance of one or more Barrel-Backed Securities 488. One or more Tokenized Representations 466 (for example, and without limitation, one or more NFTs) may provide digital representations of physical assets that are algorithmically linked to the verified state of the physical assets as recorded on the Immutable Ledger 456.

The Blockchain Layer 403 may include Loss Records 468 comprising verified records of product loss events including one or more of evaporation, leakage, or theft detected by the sensor system and recorded on the Immutable Ledger 456. Loss Records 468 may be used for Automated Tax and Loss Documentation 494. Evaporation Data 470 may comprise verified measurements of evaporative loss over time, which may be used for one or more of inventory management, tax compliance, or sustainability credit generation. Evaporation Data 470 may be used to generate one or more Angel's Share Loss Credits 496. A Chain of Custody 472 component may provide an end-to-end record of asset handling and state changes from production through distribution, enabling verification of provenance and regulatory compliance. Smart Contracts 460 may Trigger Events 474 resulting in auto-triggered payouts on sensor events.

The Blockchain-Enabled Ecosystem Architecture 400 includes an InsurTech Module 405 that leverages the trusted data from the Blockchain Layer 403 to enable novel insurance products and risk management capabilities. Parametric Insurance 476 within the InsurTech Module 405 may provide insurance products where payouts are automatically triggered based on predefined parameters recorded on the Immutable Ledger 456 rather than traditional claims adjustment processes. In certain embodiments, the Smart Contracts 460 for Parametric Insurance 476 execute payouts in a stablecoin cryptocurrency when trigger conditions are met, enabling instant settlement without currency conversion delays or intermediary processing. The trigger conditions for Parametric Insurance 476 may include one or more of contamination detection exceeding a threshold concentration, fluid level changes indicating leakage, or environmental conditions exceeding acceptable ranges.

Dynamic Risk Scoring 478 within the InsurTech Module 405 may provide real- time assessment of risk associated with monitored assets based on continuous sensor data. The Dynamic Risk Scoring 478 may update risk scores in real-time as new sensor data is recorded on the Immutable Ledger 456, enabling dynamic adjustment of insurance premiums or credit ratings based on actual asset conditions rather than static actuarial tables. Automated Loss Quantification 480 may provide automated determination of whether a loss event meets policy criteria for payout, using verified sensor data and predictions recorded on the Immutable Ledger 456 to eliminate disputes regarding loss occurrence and magnitude.

In some aspects, the InsurTech Module 405 may further include a Predictive Loss Modeling capability that trains a machine learning model on historical loss event data recorded on the Immutable Ledger 456 across a portfolio of monitored containers to predict total expected portfolio loss over a future time period. The Predictive Loss Modeling capability may aggregate historical sensor data, contamination event records, evaporation loss data, and environmental condition data from a plurality of monitored containers to identify patterns and correlations that affect portfolio-level loss rates. In some aspects, the Predictive Loss Modeling capability may generate a probability distribution of expected losses for a defined portfolio of containerized assets over a specified future period, enabling insurers and asset managers to set reserves, price policies, and make portfolio allocation decisions based on sensor-verified data rather than historical industry averages. The Predictive Loss Modeling capability may update the predicted loss distribution in real time as new sensor data is recorded on the Immutable Ledger 456, providing a dynamic, continuously refined actuarial model that reflects the current state of the monitored asset portfolio. In some aspects, the Predictive Loss Modeling capability may identify specific containers or container subpopulations within the portfolio that contribute disproportionately to predicted losses, enabling targeted inspection, remediation, or risk mitigation actions.

A Fintech Module 404 within the Blockchain-Enabled Ecosystem Architecture 400 leverages the trusted data infrastructure to enable financial products and services based on verified asset state information. Lending Contracts 482 within the Fintech Module 404 may provide smart contract-based lending arrangements where loan terms are automatically enforced based on collateral state data recorded on the Immutable Ledger 456. In certain embodiments, Lending Contracts 482 may be used for Automated Collateral Valuation 490. Asset Tokens 484 may comprise digital tokens representing ownership interests in physical assets, where the token value is algorithmically tied to the verified state of the underlying physical assets. In certain embodiments, the Asset Tokens 484 comprise non-fungible tokens (NFTs) representing ownership of specific physical assets where the NFT metadata is dynamically updated to reflect the current state of the physical asset based on real-time blockchain-verified sensor data from the Multi-Modal Sensor Array 424.

Automated Tax & Loss Documentation 494 within the Fintech Module 404 may provide automated generation of tax compliance documentation based on Loss Records 468 recorded on the Immutable Ledger 456. In certain embodiments, the Automated Tax & Loss Documentation 494 automatically calculates tax credits based on evaporation loss data recorded on the Immutable Ledger 456 and generates documentation compliant with Alcohol and Tobacco Tax and Trade Bureau (TTB) regulatory requirements for spirits industry applications.

Provenance Records 486 may provide verified records of asset origin, handling, and state changes that enable authentication and anti-counterfeiting applications. Provenance Records 486 may be used to generate Provenance Credits 499. Barrel-Backed Securities 488 may comprise financial instruments where the underlying collateral comprises aging spirits in barrels, with the security value tied to verified fill level, proof, and maturation state recorded on the Immutable Ledger 456. Automated Collateral Valuation 490 may provide real-time valuation of collateralized assets based on verified sensor data, enabling dynamic margin requirements and automated margin calls when collateral value falls below contractually defined thresholds. Tokenized Ownership 492 may provide fractional ownership of physical assets through divisible and transferable digital tokens, enabling investment in assets such as aging spirits barrels by multiple parties.

In certain embodiments, the Fintech Module 404 provides cross-industry collateral monitoring where a unified system generates real-time valuations for different types of collateralized assets across multiple industries recorded on a single blockchain network. The cross-industry collateral monitoring may enable financial institutions to manage diverse collateral portfolios including one or more of aging spirits in barrels, strategic fuel reserves in tanks, or pharmaceutical inventory in storage facilities using a common data infrastructure and valuation framework provided by the Blockchain Layer 403.

A Sustainability Credits Module 406 within the Blockchain-Enabled Ecosystem Architecture 400 enables verification and tokenization of environmental benefits and regulatory compliance based on verified sensor data. Angel's Share Loss Credits 496 within the Sustainability Credits Module 406 may comprise tradeable credits representing verified evaporative loss from aging spirits barrels, which may be used for one or more of tax deductions or trading on specialized marketplaces. Maturation Credits 497 may comprise tradeable digital tokens representing verified maturation milestones achieved by aging products. In certain embodiments, the Maturation Credits 497 may be generated when an aging product reaches a predefined maturation milestone as determined by sensor data from the Multi-Modal Sensor Array 424 and the determination is immutably recorded on the Immutable Ledger 456. The predefined maturation milestones may include one or more of chemical composition thresholds, aging duration targets, or flavor profile characteristics detected through dielectric fingerprinting.

ESG Reporting 498 within the Sustainability Credits Module 406 may provide automated environmental, social, and governance reporting based on verified sensor data and operational metrics. In certain embodiments, the ESG Reporting 498 automatically calculates ESG metrics including one or more of energy consumption per unit or product loss rates from sensor data collected by the Multi-Modal Sensor Array 424 and generates compliance reports verifiable via the Immutable Ledger 456. The automated ESG metric calculation may reduce manual data collection and reporting burden while providing third-party verifiable sustainability claims. Provenance Credits 499 may comprise tradeable credits representing verified origin and handling of products, enabling premium pricing for products with authenticated provenance recorded on the Immutable Ledger 456.

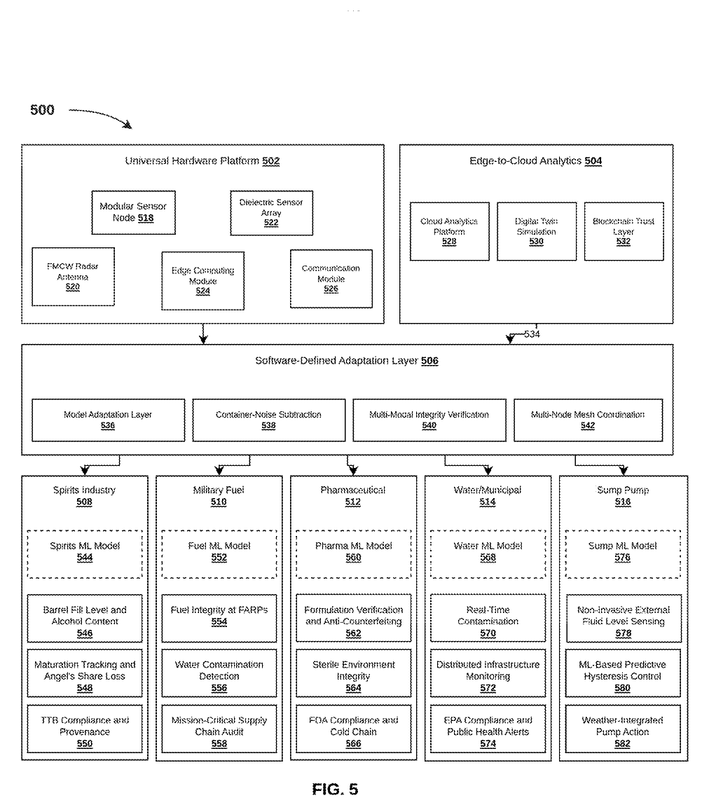

Referring to FIG. 5, a Multi-Industry Universal Platform Architecture 500 is illustrated as a layered architecture diagram depicting the software-defined platform that enables a single hardware configuration to serve multiple disparate industries through the loading of different pre-trained machine learning models. The Multi-Industry Universal Platform Architecture 500 may decouple the hardware from the application, enabling adaptation to different container types, liquid formulations, or industry-specific requirements without hardware redesign.

A Universal Hardware Platform 502 forms the hardware foundation of the Multi- Industry Universal Platform Architecture 500 and contains a Modular Sensor Node 518 that provides the physical sensing capabilities for noninvasive container monitoring. An FMCW Radar Antenna 520 within the Universal Hardware Platform 502 may provide frequency- modulated continuous wave radar sensing for precise, noninvasive fluid level detection through container walls. A Dielectric Sensor Array 522 within the Universal Hardware Platform 502 may provide electromagnetic interrogation capabilities for measuring the dielectric properties of container contents across multiple frequencies, enabling construction of dielectric fingerprints for content characterization and contamination detection.

With continued reference to FIG. 5, an Edge Computing Module 524 within the Universal Hardware Platform 502 may provide local processing capabilities for real-time machine learning inference at the sensor node. The Edge Computing Module 524 may execute compressed and quantized machine learning models to generate predictions without requiring cloud connectivity, thereby reducing latency and bandwidth requirements. A Communication Module 526 within the Universal Hardware Platform 502 may provide wireless connectivity for transmitting sensor data and predictions to cloud infrastructure and for receiving model updates and configuration commands. The Communication Module 526 may support multiple wireless communication standards including one or more of Bluetooth Low Energy, Wi-Fi, LoRaWAN, or SG cellular connectivity.

An Edge-To-Cloud Analytics 504 section of the Multi-Industry Universal Platform Architecture 500 provides the cloud-based infrastructure that supports the distributed sensor network. The Edge-To-Cloud Analytics 504 may include a Cloud Analytics Platform 528 that aggregates data from multiple sensor nodes across a distributed monitoring network, providing fleet-wide visibility, analytics dashboards, and centralized management capabilities. A Digital Twin Simulation 530 within the Edge-To-Cloud Analytics 504 may provide the virtual simulation environment for generating synthetic training data as described with reference to the 3D virtual simulation environment 300 and the Digital Twin Simulation Environment 440. A Blockchain Trust Layer 532 within the Edge-To-Cloud Analytics 504 may provide the immutable data recording capabilities described with reference to the Blockchain Layer 403, enabling creation of tamper-proof audit trails and supporting novel business models including one or more of parametric insurance products, asset-backed financing arrangements, or verifiable sustainability credits.

As further shown in FIG. 5, Model Updates & Synthetic Training Data 534 may flow from the Edge-To-Cloud Analytics 504 down to the Software-Defined Adaptation Layer 506. The Model Updates & Synthetic Training Data 534 may enable continuous improvement of deployed models based on new training data generated by the Digital Twin Simulation 530 or collected from real-world sensor deployments across the distributed network.

A Software-Defined Adaptation Layer 506 forms the intelligence layer of the Multi-Industry Universal Platform Architecture 500 and provides the capabilities that enable the Universal Hardware Platform 502 to serve multiple disparate industries through software configuration rather than hardware modification. The Software-Defined Adaptation Layer 506 may include a Model Adaptation Layer 536 that utilizes transfer learning techniques to adapt base machine learning models for specific substrates or applications. In certain embodiments, the Model Adaptation Layer 536 may refine a general hydrocarbon model trained on synthetic data representing a range of hydrocarbon fuels to create a substrate-specific model for JP-8 jet fuel that recognizes the specific dielectric signature of JP-8 and common contaminants associated with JP-8.

With continued reference to FIG. 5, a Dynamic Container-Noise Subtraction Module 538 within the Software-Defined Adaptation Layer 506 may isolate the dielectric signature of the liquid from electromagnetic contributions of the container wall material. The Dynamic Container-Noise Subtraction Module 538 may perform calibration measurements to determine electromagnetic characteristics of the container wall material and subtract the container wall contribution from liquid measurements to improve characterization accuracy. In certain embodiments, the Dynamic Container-Noise Subtraction Module 538 may account for variations in wood moisture content when monitoring spirits in wooden barrels, where the subtraction is performed by a machine learning model trained on synthetic data from the Digital Twin Simulation 530 that models both the wood and the liquid.

A Synchronous Multi-Modal Integrity Verification 540 module within the Software-Defined Adaptation Layer 506 may fuse data from the FMCW Radar Antenna 520 and the Dielectric Sensor Array 522 to provide comprehensive integrity analysis. The Synchronous Multi-Modal Integrity Verification 540 may perform temporally synchronized measurements using both sensing modalities to detect volume-neutral integrity events where a physical property such as fluid level remains substantially unchanged but a compositional property has changed. In certain embodiments, the Synchronous Multi-Modal Integrity Verification 540 may detect dilution of a spirit with water by identifying a change in permittivity measured by the Dielectric Sensor Array 522 without a corresponding change in fluid level measured by the FMCW Radar Antenna 520.

A Multi-Node Mesh Coordination 542 module within the Software-Defined Adaptation Layer 506 may coordinate anomaly detection across multiple sensor nodes in a distributed monitoring network. The Multi-Node Mesh Coordination 542 may receive anomaly signals from sensor nodes forming a mesh network and determine whether an anomaly detected by a single node is a genuine event or an environmental artifact by correlating the signal with signals from neighboring nodes. The Multi-Node Mesh Coordination 542 may classify an anomaly as an environmental event if a majority of neighboring nodes report a similar shift simultaneously, indicating that the shift is caused by an environmental factor such as a temperature change affecting multiple containers in the same area. The Multi-Node Mesh Coordination 542 may classify an anomaly as a genuine contamination event if only a single node reports the shift while neighboring nodes do not report similar deviations, indicating that the anomaly is specific to the container monitored by that single node rather than a widespread environmental effect.

With continued reference to FIG. 5, the Multi-Industry Universal Platform Architecture 500 includes five Industry Verticals at the bottom of the architecture diagram, each representing a distinct application domain that can be served by the Universal Hardware Platform 502 through the loading of a corresponding pre-trained machine learning model via the Software-Defined Adaptation Layer 506. The five Industry Verticals demonstrate the software-defined versatility of the platform, where the same hardware configuration may be adapted to disparate industries by deploying different machine learning models that have been trained on industry-specific synthetic data generated by the Digital Twin Simulation 530. The five Industry Verticals are given by way of illustration rather than limitation; the technology disclosed herein may be applied to any suitable industry, and as such, other industry verticals may be used without departing from the scope of the present disclosure.

A Spirits Industry Factors 508 vertical represents the application of the platform to monitoring aging spirits in barrels, including one or more of whiskey, bourbon, or wine. The Spirits Industry Factors 508 vertical may utilize a Spirits ML Model 544 that has been trained on synthetic data representing the electromagnetic interactions between radar signals, dielectric interrogation signals, and spirits aging in wooden barrels. The Spirits ML Model 544 may be configured to predict multiple properties of the barrel contents based on sensor data from the FMCW Radar Antenna 520 and the Dielectric Sensor Array 522.

A Barrel Fill Level and Alcohol Content 546 capability within the Spirits Industry Factors 508 vertical may provide predictions of the current fill level within the barrel and the alcohol proof of the spirits. The Barrel Fill Level and Alcohol Content 546 capability may utilize FMCW radar measurements to determine the fluid level through the wooden barrel wall and dielectric fingerprinting to characterize the alcohol-water ratio based on the frequency- dependent permittivity of the spirits. In certain embodiments, the Barrel Fill Level and Alcohol Content 546 capability detects changes in proof over time as the spirits interact with the wood and undergo evaporation.

A Maturation Tracking and Angel's Share Loss 548 capability within the Spirits Industry Factors 508 vertical may provide predictions regarding the aging state of the spirits and quantification of evaporative loss over time. The Maturation Tracking and Angel's Share Loss 548 capability may track chemical composition changes that occur during aging by analyzing shifts in the dielectric fingerprint that correlate with flavor profile development. The evaporative loss quantification provided by the Maturation Tracking and Angel's Share Loss 548 capability may be recorded on the Immutable Ledger 456 and used to generate the Angel's Share Loss Credits 496 described with reference to the Sustainability Credits Module 406.

A TTB Compliance and Provenance 550 capability within the Spirits Industry Factors 508 vertical may provide automated regulatory compliance documentation and verifiable chain of custody records for spirits industry applications. The TTB Compliance and Provenance 550 capability may generate documentation compliant with Alcohol and Tobacco Tax and Trade Bureau regulations based on verified sensor data recorded on the Immutable Ledger 456. The TTB Compliance and Provenance 550 capability may utilize the Provenance Records 486 from the Fintech Module 404 to provide an end-to-end record of barrel handling and state changes from production through distribution.

A Military Fuel Factors 510 vertical represents the application of the platform to monitoring strategic fuel reserves in military logistics applications. The Military Fuel Factors 510 vertical may utilize a Fuel ML Model 552 that has been trained on synthetic data representing the electromagnetic interactions between sensor signals and hydrocarbon fuels including one or more of JP-8 jet fuel, diesel, or aviation gasoline stored in metal tanks. The Fuel ML Model 552 may be adapted from a general hydrocarbon model using the Model Adaptation Layer 536 to recognize the specific dielectric signature of the target fuel type and common contaminants associated with that fuel type.

A Fuel Integrity at FARPs 554 capability within the Military Fuel Factors 510 vertical may provide monitoring of fuel quality at Forward Arming and Refueling Points where aircraft receive fuel in field conditions. The Fuel Integrity at FARPs 554 capability may continuously verify that fuel meets quality specifications before being dispensed to aircraft, which may be relevant for mission success and operational safety in military aviation applications.

A Water Contamination Detection 556 capability within the Military Fuel Factors 510 vertical may provide detection of water ingress in fuel storage tanks. The Water Contamination Detection 556 capability may utilize the dielectric fingerprinting capabilities of the Dielectric Sensor Array 522 to detect water contamination based on the high permittivity of water relative to hydrocarbon fuels. In certain embodiments, the Water Contamination Detection 556 capability may detect water concentrations below thresholds that would cause operational problems, enabling remediation before the contamination affects fuel quality.

A Mission-Critical Supply Chain Audit 558 capability within the Military Fuel Factors 510 vertical may provide verifiable records of fuel handling and state throughout the military supply chain. The Mission-Critical Supply Chain Audit 558 capability may utilize the Chain of Custody 472 component of the Blockchain Layer 403 to create an immutable record of fuel provenance and integrity from refinery through distribution to point of use.

A Pharmaceutical Factors 512 vertical represents the application of the platform to monitoring liquid pharmaceuticals in manufacturing and distribution applications. The Pharmaceutical Factors 512 vertical may utilize a Pharma Model 560 that has been trained on synthetic data representing the electromagnetic interactions between sensor signals and pharmaceutical formulations stored in one or more of glass vials, plastic containers, or stainless steel vessels.

A Formulation Verification and Anti-Counterfeiting 562 capability within the Pharmaceutical Factors 512 vertical may provide verification that pharmaceutical formulations match expected compositions and detection of counterfeit or adulterated products. The Formulation Verification and Anti-Counterfeiting 562 capability may utilize dielectric fingerprinting to compare the measured dielectric spectrum of a pharmaceutical product against a reference spectrum for the authentic formulation, with deviations indicating potential counterfeiting or formulation errors.

A Sterile Environment Integrity 564 capability within the Pharmaceutical Factors 512 vertical may provide monitoring of environmental conditions that affect pharmaceutical product quality and sterility. The Sterile Environment Integrity 564 capability may utilize environmental sensor data from the Multi-Modal Sensor Array 424 to verify that storage conditions remain within acceptable ranges for maintaining product sterility and efficacy.

An FDA Compliance and Cold Chain 566 capability within the Pharmaceutical Factors 512 vertical may provide automated regulatory compliance documentation and verification of temperature-controlled distribution for pharmaceutical products. The FDA Compliance and Cold Chain 566 capability may generate documentation compliant with Food and Drug Administration regulations based on verified sensor data recorded on the Immutable Ledger 456. The FDA Compliance and Cold Chain 566 capability may track temperature excursions during distribution and provide an unbroken, verifiable record of handling for temperature-sensitive liquid medicines.

A Water/Municipal Factors 514 vertical represents the application of the platform to monitoring water quality in municipal water treatment and distribution applications. The Water/Municipal Factors 514 vertical may utilize a Water ML Model 568 that has been trained on synthetic data representing the electromagnetic interactions between sensor signals and potable water containing various potential contaminants including one or more of chemical pollutants, biological agents, or industrial solvents.

A Real-Time Contamination Detection 570 capability within the Water/Municipal Factors 514 vertical may provide continuous monitoring for contaminants in water distribution systems. The Real-Time Contamination Detection 570 capability may utilize dielectric fingerprinting to detect deviations from baseline water quality that indicate the presence of contaminants, enabling early warning of contamination events before the contaminants reach consumers.

A Distributed Infrastructure Monitoring 572 capability within the Water/Municipal Factors 514 vertical may provide monitoring across geographically distributed water infrastructure including one or more of treatment plants, storage tanks, or distribution pipelines. The Distributed Infrastructure Monitoring 572 capability may utilize the Multi-Node Mesh Coordination 542 to correlate anomaly signals across multiple sensor nodes and distinguish between localized contamination events and widespread environmental effects.

An EPA Compliance and Public Health Alerts 574 capability within the Water/Municipal Factors 514 vertical may provide automated regulatory compliance documentation and public notification capabilities for water quality events. The EPA Compliance and Public Health Alerts 574 capability may generate documentation compliant with Environmental Protection Agency regulations based on verified sensor data recorded on the Immutable Ledger 456. The EPA Compliance and Public Health Alerts 574 capability may automatically generate public health alerts when contamination levels exceed regulatory thresholds, enabling rapid response to protect public health.

A Sump Factors 516 vertical represents the application of the platform to monitoring and controlling residential and commercial sump pump systems. The Sump Factors 516 vertical may utilize a Sump ML Model 576 that has been trained on synthetic data representing the electromagnetic interactions between radar signals and water in plastic or composite sump basins.

A noninvasive External Fluid Level Sensing 578 capability within the Sump Factors 516 vertical may provide fluid level measurement through the sump basin wall without physical contact with the water. The noninvasive External Fluid Level Sensing 578 capability may utilize the FMCW Radar Antenna 520 to measure water level through the basin material, eliminating the mechanical failure modes associated with traditional float switches including one or more of debris accumulation, corrosion, or mechanical wear.

An ML-Based Predictive Hysteresis Control 580 capability within the Sump Factors 516 vertical may provide intelligent pump activation that goes beyond simple high/low threshold control. The ML-Based Predictive Hysteresis Control 580 capability may incorporate the current fluid level, the rate of change of the fluid level, and historical patterns to determine optimal pump activation timing that prevents both flooding and excessive pump cycling.

A Weather-Integrated Pump Activation 582 capability within the Sump Factors 516 vertical may incorporate external weather forecast data into pump control decisions. The Weather-Integrated Pump Activation 582 capability may proactively lower the water level in the basin when heavy rainfall is predicted, increasing the basin's capacity to handle the anticipated inflow and preventing the pump from being overwhelmed during storm events.

In certain embodiments, the Multi-Industry Universal Platform Architecture 500 may provide multi-regulatory provenance capabilities where a provenance module generates verifiable end-to-end chain of custody records that are compliant with regulatory requirements across different industries. The provenance module may generate chain of custody records compliant with TTB regulations for spirits industry applications monitored using the Spirits ML Model 544 and chain of custody records compliant with FDA regulations for pharmaceutical applications monitored using the Pharma Model 560. The multi-regulatory provenance capabilities may utilize the Provenance Records 486 from the Fintech Module 404 and the Chain of Custody 472 component from the Blockchain Layer 403 to provide a unified provenance infrastructure that adapts documentation formats and compliance requirements based on the industry vertical being served while maintaining a common underlying data structure recorded on the Immutable Ledger 456.

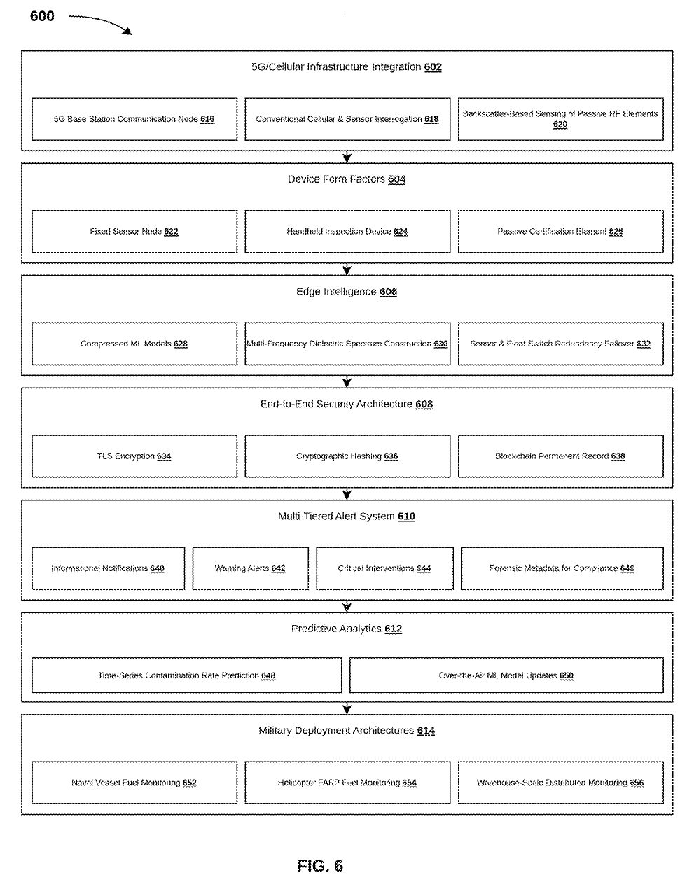

Referring to FIG. 6, an Advanced Deployment and Infrastructure Architecture 600 is illustrated as a vertical flowchart depicting the platform's deployment strategies, infrastructure integration capabilities, and operational architectures organized into seven example sequential sections. The Advanced Deployment and Infrastructure Architecture 600 may extend the capabilities of the Multi-Industry Universal Platform Architecture 500 by providing specific configurations for integrating with existing telecommunications infrastructure, supporting multiple device form factors, and enabling advanced edge intelligence capabilities.

A 5G/Cellular Infrastructure Integration 602 section forms the first section of the Advanced Deployment and Infrastructure Architecture 600 and provides capabilities for leveraging existing cellular network infrastructure for dual-use communication and sensing applications. The 5G/Cellular Infrastructure Integration 602 section may enable the platform to utilize wireless communication nodes that are already deployed for cellular communication services to perform container monitoring functions, thereby reducing infrastructure deployment costs and leveraging existing network coverage.

With continued reference to FIG. 6, a 5G Base Station Wireless Communication Node 616 within the 5G/Cellular Infrastructure Integration 602 may comprise a gNodeB base station or other cellular network node that provides both conventional cellular communication services and sensor interrogation capabilities. The 5G Base Station Wireless Communication Node 616 may include an antenna array and radio frequency front end that directs beamformed RF signals towards monitored containers equipped with RF-responsive sensor elements. In certain embodiments, the 5G Base Station Wireless Communication Node 616 uses time/frequency division to dynamically allocate time slots between conventional cellular communication functions and sensor interrogation functions. The time/frequency division approach may interleave communication time slots with sensing time slots such that the 5G Base Station Wireless Communication Node 616 handles conventional cellular communication during designated communication time slots and performs sensor interrogation during interleaved sensing time slots.

A Conventional Cellular and Sensor Interrogation 618 capability within the 5G/Cellular Infrastructure Integration 602 may provide the dual-use functionality that enables the 5G Base Station Wireless Communication Node 616 to serve both communication and sensing purposes. The Conventional Cellular and Sensor Interrogation 618 capability may utilize an RF transceiver within the 5G Base Station Wireless Communication Node 616 to handle conventional cellular communication during communication time slots while a sensor interrogation module uses the antenna array to send interrogation signals and analyze reflected signals during sensing time slots. The dual-use architecture provided by the Conventional Cellular and Sensor Interrogation 618 capability may effectively transform a standard 5G base station into a wide-area, multi-container monitoring system without requiring dedicated sensing infrastructure.

A Backscatter-Based Sensing of Passive RF-Response Elements 620 capability within the 5G/Cellular Infrastructure Integration 602 may enable interrogation of passive sensor elements affixed to containers without requiring active electronics or power sources on the container. The Backscatter-Based Sensing of Passive RF-Response Elements 620 capability may transmit interrogation signals from the 5G Base Station Wireless Communication Node 616 towards containers equipped with passive RF-responsive elements and analyze the backscattered signals to determine properties of the container contents. In certain embodiments, the backscattered signal may be modulated by an interaction between the passive element's antenna and the dielectric properties of the liquid inside the container, enabling characterization of container contents based on the backscatter characteristics.

As further shown in FIG. 6, a Device Form Factors 604 section forms the second section of the Advanced Deployment and Infrastructure Architecture 600 and illustrates three physical form options for deploying the sensing capabilities of the platform. The Device Form Factors 604 may enable selection of an appropriate physical configuration based on the deployment requirements of a particular application, ranging from permanently installed monitoring nodes to portable inspection devices to passive certification elements.

A Fixed Sensor Node 622 of the Device Form Factors 604 may comprise a permanently installed sensor unit that is attached to a container for continuous monitoring. The Fixed Sensor Node 622 may correspond to the Modular Sensor Node 518 described with reference to the Multi-Industry Universal Platform Architecture 500 and may include the FMCW Radar Antenna 520, the Dielectric Sensor Array 522, the Edge Computing Module 524, and the Communication Module 526. The Fixed Sensor Node 622 may be suitable for applications requiring continuous, long-term monitoring of containers such as aging barrels in a rickhouse or fuel tanks at a storage facility.

A Handheld Inspection Device 624 of the Device Form Factors 604 may comprise a portable, handheld device for noninvasive container inspection that can be carried by a user to perform spot checks or inspections of containers that are not equipped with fixed monitoring nodes. The Handheld Inspection Device 624 may include a housing configured to be held by a user, a multi-modal sensor array integrated within the housing, an edge computing module within the housing configured to execute a machine learning model, and a display on the housing for presenting predictions about container contents generated by the machine learning model. In certain embodiments, the Handheld Inspection Device 624 may be configured to load different machine learning models for inspecting different types of containers, enabling a single handheld device to be used across multiple industry verticals by loading the appropriate model for the container type being inspected.

A Passive Certification Element 626 of the Device Form Factors 604 may comprise a passive element affixed to an external surface of a container that has an electromagnetic response that changes based on a property of a liquid inside the container. The Passive Certification Element 626 may be interrogated by one or more of the Fixed Sensor Node 622, the Handheld Inspection Device 624, or the 5G Base Station Wireless Communication Node 616 to verify the integrity of the container contents without requiring active electronics or power sources on the container. In certain embodiments, the Passive Certification Element 626 comprises a chipless RFID tag whose resonant frequency shifts in response to changes in the dielectric constant of the liquid in close proximity to the tag. The chipless RFID tag configuration of the Passive Certification Element 626 may provide a low-cost, maintenance- free certification mechanism that can be affixed to containers during manufacturing and interrogated throughout the container's lifecycle to verify content integrity.

With continued reference to FIG. 6, an Edge Intelligence 606 section forms the third section of the Advanced Deployment and Infrastructure Architecture 600 and contains capabilities for performing intelligent processing at the sensor node level without requiring cloud connectivity. The Edge Intelligence 606 may enable real-time inference and decision- making at the point of data collection, reducing latency and bandwidth requirements while maintaining the ability to operate in environments with limited or intermittent network connectivity.

A Compressed ML Models 628 capability within the Edge Intelligence 606 may provide machine learning models that have been optimized for execution on resource- constrained edge devices. The Compressed ML Models 628 may be created by applying model compression or quantization techniques to full-precision machine learning models trained on server or cloud platforms, resulting in compressed model variants with smaller memory footprints and lower computational requirements suitable for deployment on the Edge Computing Module 524. In certain embodiments, the model compression technique may include 8-bit integer quantization to create a compressed model variant, where the 8-bit integer quantization reduces the precision of model weights and activations from 32-bit floating point to 8-bit integers, thereby reducing memory requirements and enabling faster inference on edge devices with limited computational resources.

A Multi-Frequency Dielectric Spectrum Construction 630 capability within the Edge Intelligence 606 may provide the ability to construct a complete dielectric spectrum of a liquid by interrogating the liquid with electromagnetic signals at multiple frequencies across a predefined spectrum. The Multi-Frequency Dielectric Spectrum Construction 630 capability may measure the dielectric property of the liquid at each of the different frequencies and construct a dielectric spectrum that serves as a unique fingerprint for the liquid. The dielectric spectrum constructed by the Multi-Frequency Dielectric Spectrum Construction 630 capability may be compared to a library of known spectra using a machine learning model executed by the Compressed ML Models 628 to identify the liquid or detect contamination based on deviations from expected spectral characteristics.

A noninvasive Sensor and Float Switch Redundancy Automatic Failover 632 capability within the Edge Intelligence 606 may provide dual-mode operation that combines noninvasive sensing with traditional float switch sensing for applications requiring maximum reliability. The noninvasive Sensor and Float Switch Redundancy Automatic Failover 632 capability may operate in a dual-mode configuration where the system uses one of the noninvasive sensor or a float switch as a primary sensor and the other as a backup, with automatic detection of failure in the primary sensor and failover to the backup sensor. In certain embodiments, the noninvasive sensor is primary and the float switch is backup, and a failure is detected when the noninvasive sensor provides a reading that is statistically improbable or inconsistent with a historical rate of change. The noninvasive Sensor and Float Switch Redundancy Automatic Failover 632 capability may be particularly applicable to the Sump Factors 516 vertical where the consequences of sensor failure can include flooding, and the dual-mode configuration provides a fail-safe mechanism that maintains pump control even if one sensing modality fails.

With continued reference to FIG. 6, an End-To-End Security Architecture 608 section forms the fourth section of the Advanced Deployment and Infrastructure Architecture 600 and provides a comprehensive security chain that ensures data integrity from the point of sensor data acquisition through permanent recording on a distributed ledger. The End-To-End Security Architecture 608 may create a tamper-proof forensic record that is available to authorized stakeholders and provides confidence in the integrity of sensor data throughout the data lifecycle.

A TLS Encryption 634 capability within the End-To-End Security Architecture 608 may provide transport layer security for data transmitted between sensor nodes and cloud infrastructure. The TLS Encryption 634 may encrypt communication channels between the Edge Computing Module 524 and the Cloud Analytics Platform 528, protecting sensor data and predictions from interception or modification during transmission. In certain embodiments, the TLS Encryption 634 may establish encrypted sessions using certificate-based authentication to verify the identity of communicating parties before data exchange occurs.

A Cryptographic Hashing 636 capability within the End-To-End Security Architecture 608 may generate cryptographic hashes of sensor data at the point of data acquisition within the sensor node. The Cryptographic Hashing 636 may be performed by a hardware security module embedded within the Edge Computing Module 524, ensuring that the hash is generated at the moment of data creation before the data leaves the sensor node. The cryptographic hash generated by the Cryptographic Hashing 636 capability may serve as a digital fingerprint of the sensor data that can be used to verify data integrity at any subsequent point in the data lifecycle. In certain embodiments, the Cryptographic Hashing 636 may generate a hash using a secure hashing algorithm that produces a fixed-length output from variable-length input data, where any modification to the original sensor data would result in a different hash value, thereby enabling detection of tampering.

A Blockchain Permanent Record 638 capability within the End-To-End Security Architecture 608 may record the cryptographic hash generated by the Cryptographic Hashing 636 on the Immutable Ledger 456 of the Blockchain Layer 403. The Blockchain Permanent Record 638 may create a permanent, distributed record of the hash that is resistant to alteration without detection, thereby providing a verifiable proof of data integrity that persists indefinitely. The combination of the TLS Encryption 634, the Cryptographic Hashing 636, and the Blockchain Permanent Record 638 may create an end-to-end security chain where sensor data is secured at the point of collection, protected during transmission, and permanently recorded in a tamper-proof manner, enabling verification of data integrity from sensor to submission for regulatory compliance or audit purposes.

As further shown in FIG. 6, a Multi-Tiered Alert System 610 section forms the fifth section of the Advanced Deployment and Infrastructure Architecture 600 and provides a hierarchical alerting framework that generates notifications of varying severity based on the nature and magnitude of detected anomalies. The Multi-Tiered Alert System 610 may enable appropriate response escalation based on the severity of detected conditions, ranging from informational notifications for minor deviations to critical alerts requiring immediate intervention.

An Informational Notifications 640 tier within the Multi-Tiered Alert System 610 may comprise alerts generated for minor deviations from expected conditions that do not require immediate action but may be of interest for operational awareness or trend analysis. The Informational Notifications 640 may include one or more of routine status updates, minor parameter variations within acceptable ranges, or notifications of scheduled maintenance requirements.

A Warning Alerts 642 tier within the Multi-Tiered Alert System 610 may comprise alerts generated for moderate deviations from expected conditions that warrant attention and may require corrective action if the condition persists or worsens. The Warning Alerts 642 may include one or more of parameter values approaching threshold limits, gradual trends indicating potential future problems, or conditions that deviate from baseline but remain within operational tolerances.

A Critical Interventions 644 tier within the Multi-Tiered Alert System 610 may comprise alerts generated for severe deviations from expected conditions that require immediate intervention to prevent damage, loss, or safety hazards. The Critical Interventions 644 may include one or more of contamination detection exceeding safety thresholds, rapid fluid level changes indicating leakage or theft, or sensor readings indicating imminent equipment failure.

A Forensic Metadata for Regulators Compliance and Audit 646 capability within the Multi-Tiered Alert System 610 may automatically include detailed metadata with each generated alert to support regulatory compliance and audit requirements. The Forensic Metadata for Regulators Compliance and Audit 646 may comprise the sensor data that triggered the alert, the AI prediction generated by the machine learning model, a confidence score indicating the model's certainty in the prediction, and a timestamp indicating when the alert condition was detected. The forensic metadata included by the Forensic Metadata for Regulators Compliance and Audit 646 capability may be suitable for regulatory compliance submissions and audit documentation, providing a complete record of the conditions that led to alert generation and the basis for any automated or manual response actions taken.

With continued reference to FIG. 6, a Predictive Analytics 612 section forms the sixth section of the Advanced Deployment and Infrastructure Architecture 600 and provides capabilities for forecasting future conditions based on historical sensor data and for maintaining deployed models with updated capabilities. The Predictive Analytics 612 may enable proactive response to anticipated conditions rather than reactive response to conditions that have already occurred.

A Time-Series Contamination Rate Prediction 648 capability within the Predictive Analytics 612 may analyze time-series data comprising dielectric property measurements collected from a liquid within a container over a period of time to determine a rate of change of the dielectric property and predict future contamination levels or time-to-failure based on the rate of change. In certain embodiments, the Time-Series Contamination Rate Prediction 648 inputs the time-series data into a recurrent neural network (RNN) or LSTM model to predict the future contamination rate, enabling predictive maintenance before the contamination reaches a threshold that would require remediation or cause operational problems. The RNN or LSTM model architecture used by the Time-Series Contamination Rate Prediction 648 may capture temporal dependencies in the sensor data, enabling the model to learn patterns of contamination progression over time and extrapolate those patterns to predict future contamination states.

An Over-the-Air ML Model Updates 650 capability within the Predictive Analytics 612 may provide the ability to transmit updated machine learning models from a remote management system to deployed sensor nodes without requiring physical access to the sensor nodes. The Over-the-Air ML Model Updates 650 may establish a communication link between the Cloud Analytics Platform 528 and a sensor node via the Communication Module 526 and transmit an updated machine learning model that replaces the existing model on the sensor node, thereby remotely upgrading the sensor node's capabilities. In certain embodiments, the updated machine learning model transmitted by the Over-the-Air ML Model Updates 650 comprises a compressed model variant created using the techniques described with reference to the Compressed ML Models 628, and the transmission occurs via a 5G cellular network connection provided by the 5G Base Station Wireless Communication Node 616. The Over- the-Air ML Model Updates 650 capability may enable continuous improvement of deployed sensor nodes as new training data becomes available and improved models are developed, without requiring field service visits to update individual sensor nodes.

A Military Deployment Architectures 614 section forms the seventh section of the Advanced Deployment and Infrastructure Architecture 600 and provides specific configurations for deploying the platform in military logistics applications where fuel integrity monitoring is relevant for operational readiness and mission success. The Military Deployment Architectures 614 may address the unique requirements of military environments including one or more of harsh operating conditions, mobile deployment scenarios, or stringent security requirements.

A Naval Vessel Fuel Monitoring 652 configuration within the Military Deployment Architectures 614 may provide a deployment architecture for monitoring fuel integrity aboard naval vessels. The Naval Vessel Fuel Monitoring 652 configuration may deploy sensor nodes on fuel storage tanks within the vessel to continuously monitor fuel level and detect contamination including water ingress that could affect engine performance or cause equipment damage. The Naval Vessel Fuel Monitoring 652 configuration may utilize the Fuel ML Model 552 adapted for the specific fuel types used in naval applications and may integrate with shipboard systems for centralized monitoring and alerting.

A Helicopter FARP Fuel Monitoring 654 configuration within the Military Deployment Architectures 614 may provide a deployment architecture for monitoring fuel integrity at Forward Arming and Refueling Points where helicopters and other aircraft receive fuel in field conditions. The Helicopter FARP Fuel Monitoring 654 configuration may deploy sensor nodes on portable fuel storage containers and fuel distribution equipment to verify fuel quality before dispensing to aircraft. The Helicopter FARP Fuel Monitoring 654 configuration may utilize the Fuel Integrity at FARPs 554 capability and the Water Contamination Detection 556 capability to ensure that fuel meets quality specifications in austere field environments where contamination risks may be elevated due to one or more of dust, moisture, or handling conditions.

A Warehouse-Scale Distributed Monitoring 656 configuration within the Military Deployment Architectures 614 may provide a deployment architecture for monitoring large numbers of containers across extensive storage facilities. The Warehouse-Scale Distributed Monitoring 656 configuration may deploy sensor nodes across hundreds or thousands of containers in a warehouse or depot environment, utilizing the Multi-Node Mesh Coordination 542 to coordinate anomaly detection across the distributed sensor network and distinguish between localized events affecting individual containers and widespread environmental effects affecting multiple containers simultaneously. The Warehouse-Scale Distributed Monitoring 656 configuration may provide fleet-wide visibility into the state of stored assets and enable prioritization of inspection and maintenance activities based on sensor data indicating which containers require attention.

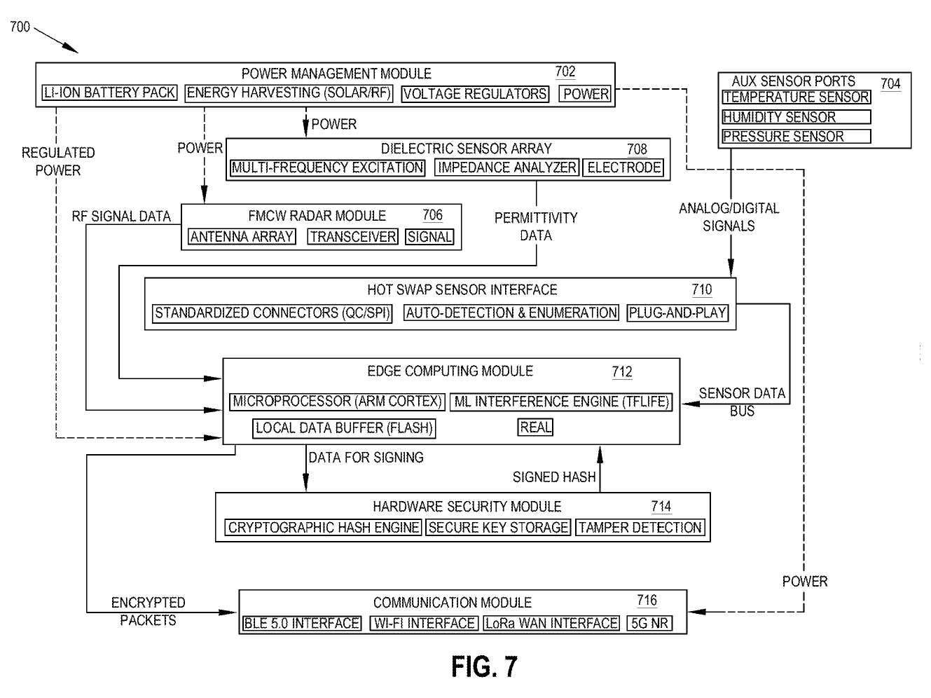

Referring to FIG. 7, a Modular Sensor Node Hardware Block Diagram 700 is illustrated as a detailed hardware block diagram depicting the physical components and interconnections of the modular sensor node that provides the sensing, processing, and communication capabilities for noninvasive container monitoring. The Modular Sensor Node Hardware Block Diagram 700 may correspond to the physical implementation of the Modular Sensor Node 518 described with reference to the Multi-Industry Universal Platform Architecture 500 and the Fixed Sensor Node 622 described with reference to the Device Form Factors 604.

A Power Management Module 702 within the Modular Sensor Node Hardware Block Diagram 700 supplies power to all other modules in the sensor node. The Power Management Module 702 may include one or more of battery power, energy harvesting capabilities, or voltage regulators to provide stable power supply to the sensing, processing, and communication components. The energy harvesting capabilities of the Power Management Module 702 may enable the sensor node to supplement battery power by harvesting energy from one or more of ambient light, thermal gradients, or vibration, thereby extending operational lifetime between battery replacements or enabling battery-free operation in environments with sufficient harvestable energy. The voltage regulators within the Power Management Module 702 may convert the input power from the battery or energy harvesting sources to the specific voltage levels used by each module within the sensor node.

With continued reference to FIG. 7, an FMCW Radar Module 706 and a Dielectric Sensor Array 708 serve as the primary sensing units within the Modular Sensor Node Hardware Block Diagram 700. The FMCW Radar Module 706 may transmit frequency-modulated continuous wave radar signals through the container wall and analyze the returned signals to determine fluid level within the container. The FMCW Radar Module 706 may send RF signal data to an Edge Computing Module 712 for processing and interpretation by machine learning models. The Dielectric Sensor Array 708 may interrogate the container contents with electromagnetic signals across multiple frequencies to measure the dielectric properties of the liquid, generating permittivity data that is sent to the Edge Computing Module 712. The permittivity data from the Dielectric Sensor Array 708 may be used to construct dielectric fingerprints for content characterization and contamination detection as described with reference to the Multi-Frequency Dielectric Spectrum Construction 630.

A Hot-Swap Sensor Interface 710 within the Modular Sensor Node Hardware Block Diagram 700 may receive analog/digital signals from one or more Auxiliary Sensor Ports 704. The Auxiliary Sensor Ports 704 may include one or more of temperature sensors, humidity sensors, or other environmental sensors that provide supplementary data to complement the primary measurements from the FMCW Radar Module 706 and the Dielectric Sensor Array 708. The Hot-Swap Sensor Interface 710 may enable field-configurable sensor arrays where auxiliary sensors can be added, removed, or replaced based on application requirements without disrupting ongoing monitoring operations. Data from the Auxiliary Sensor Ports 704 connected via the Hot-Swap Sensor Interface 710 may be fed to the Edge Computing Module 712 for integration with the primary sensor data during processing and inference operations.

In some aspects, the Auxiliary Sensor Ports 704 may accommodate sensing modalities that provide complementary contamination detection capabilities beyond the primary FMCW radar and dielectric fingerprinting measurements. In some aspects, the Auxiliary Sensor Ports 704 may receive data from a capacitance-based sensor configured to measure changes in the capacitance of the liquid within the container, where changes in capacitance correlate with changes in the dielectric constant of the liquid at a fixed frequency and may indicate the presence of a contaminant with a permittivity different from the baseline liquid. In some aspects, the Auxiliary Sensor Ports 704 may receive data from an electrochemical sensor configured to measure one or more of pH, oxidation-reduction potential, or specific ion concentration of the liquid, providing chemical characterization data that complements the electromagnetic measurements from the FMCW Radar Module 706 and the Dielectric Sensor Array 708. The Edge Computing Module 712 may fuse data from the capacitance-based sensor, the electrochemical sensor, and the primary sensing modalities using the loaded machine learning model to improve contamination classification accuracy, where the combination of electromagnetic, capacitive, and electrochemical measurements may resolve ambiguities that arise when different contaminants produce similar responses in a single sensing modality. In some aspects, the Synchronous Multi-Modal Integrity Verification 540 module may correlate temporally synchronized measurements from the capacitance-based sensor, the electrochemical sensor, and the primary FMCW radar and dielectric fingerprinting modalities to detect contamination events that would be undetectable by any single sensing modality alone.

As further shown in FIG. 7, the Edge Computing Module 712 contains the main microprocessor and machine learning inference engine that processes sensor data from the FMCW Radar Module 706, the Dielectric Sensor Array 708, and the Auxiliary Sensor Ports 704. The Edge Computing Module 712 may execute the Compressed ML Models 628 to generate predictions regarding container contents without requiring cloud connectivity, enabling real-time inference at the point of data collection. The Edge Computing Module 712 may correspond to the Edge Computing Module 524 described with reference to the Multi- Industry Universal Platform Architecture 500 and may provide the local processing capabilities described with reference to the Edge Intelligence 606.

A Hardware Security Module 714 within the Modular Sensor Node Hardware Block Diagram 700 receives processed data from the Edge Computing Module 712 and performs cryptographic signing to ensure data integrity and authenticity. The Hardware Security Module 714 may generate cryptographic hashes of sensor data at the point of acquisition as described with reference to the Cryptographic Hashing 636, creating a digital fingerprint that can be used to verify data integrity throughout the data lifecycle. The Hardware Security Module 714 may store cryptographic keys in a secure, tamper-resistant environment and perform signing operations within the secure boundary, preventing extraction of private keys even if other components of the sensor node are compromised. The signed and encrypted data packets produced by the Hardware Security Module 714 may be transmitted to cloud infrastructure for recording on the Immutable Ledger 456 via the Blockchain Permanent Record 638.

A Communication Module 716 within the Modular Sensor Node Hardware Block Diagram 700 may receive encrypted packets from the Edge Computing Module 712. The Communication Module 716 may support multiple wireless standards including one or more of Bluetooth Low Energy (BLE), Wi-Fi, LoRaWAN, or 5G cellular connectivity to provide flexible connectivity options based on deployment environment requirements. The multi- protocol wireless communication capabilities of the Communication Module 716 may enable the sensor node to utilize the most appropriate communication technology for a given deployment, with BLE being suitable for short-range communication with mobile devices, Wi- Fi being suitable for deployments with existing wireless network infrastructure, LoRaWAN being suitable for long-range, low-power communication in remote or distributed deployments, and 5G being suitable for high-bandwidth applications requiring low latency or integration with the 5G/Cellular Infrastructure Integration 602. The Communication Module 716 may correspond to the Communication Module 526 described with reference to the Multi-Industry Universal Platform Architecture 500 and may receive the Over-the-Air ML Model Updates 650 for deployment to the Edge Computing Module 712.

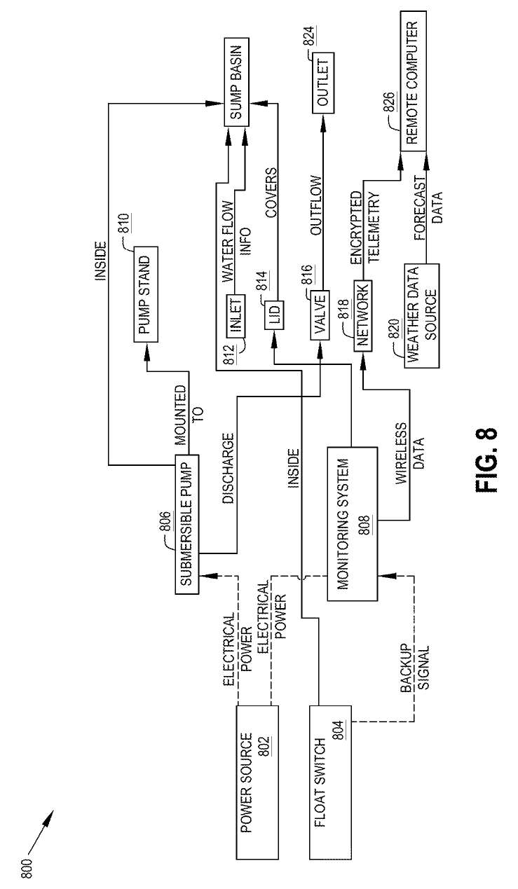

Referring to FIG. 8, a Sump Pump Monitoring System 800 is illustrated as a cross- sectional diagram depicting a complete installation of the noninvasive monitoring and control system for residential or commercial sump pump applications. The Sump Pump Monitoring System 800 may represent a physical implementation of the Sump Factors 516 vertical described with reference to the Multi-Industry Universal Platform Architecture 500, utilizing the Sump ML Model 576 to provide intelligent pump control that replaces unreliable mechanical sensing mechanisms with noninvasive, failure-resistant monitoring capabilities.

A Monitoring System 808 is externally mounted on a Lid 814 of a Sump Basin 822, positioning the sensing components outside the basin without physical contact with the water contained within the sump basin. The external mounting of the Monitoring System 808 on the Lid 814 may enable noninvasive fluid level detection through the lid material using FMCW radar sensing as described with reference to the noninvasive External Fluid Level Sensing 578 capability. The Monitoring System 808 may comprise sensing, processing, and communication components corresponding to those described with reference to the Modular Sensor Node Hardware Block Diagram 700, including the FMCW Radar Module 706 for fluid level measurement, the Edge Computing Module 712 for local inference using the Compressed ML Models 628, and the Communication Module 716 for wireless data transmission.

In some aspects, the model compression and quantization techniques applied to generate the Compressed ML Models 628 may constitute enabling technologies that are specific to the domain of dielectric fingerprint classification on resource-constrained edge devices. The model compression pipeline may include a domain-aware quantization process in which the quantization precision is varied across different layers of the neural network based on the sensitivity of each layer to quantization error in the specific context of dielectric spectrum classification. In some aspects, layers responsible for detecting subtle frequency- dependent permittivity shifts (corresponding to low-concentration contaminants) may retain higher numerical precision (e.g., 16-bit floating point) while layers responsible for gross feature extraction may be quantized more aggressively (e.g., 8-bit integer), producing a mixed precision compressed model that preserves detection sensitivity for critical contaminant thresholds while minimizing overall memory footprint. The mixed-precision quantization process may be automated by a quantization sensitivity analysis tool that evaluates the impact of quantization on detection accuracy for a representative set of contaminant scenarios generated by the Digital Twin Simulation 530.

In some aspects, the Hot-Swap Sensor Interface 710 may implement a standardized discovery and configuration protocol that enables the Edge Computing Module 712 to automatically detect the type and capabilities of a sensor connected to the Auxiliary Sensor Ports 704 without manual configuration. The discovery and configuration protocol may include an enumeration phase in which the connected sensor transmits a sensor descriptor comprising the sensing modality type, measurement range, sampling rate, and data format, followed by a configuration phase in which the Edge Computing Module 712 loads a corresponding sensor driver module and configures the data acquisition pipeline for the detected sensor type. The standardized discovery and configuration protocol may enable field technicians to upgrade or replace sensors on deployed Modular Sensor Nodes without specialized tools, software configuration, or firmware updates, reducing deployment and maintenance costs for large-scale distributed monitoring networks.

In some aspects, the Passive Certification Element 626 may employ an enabling technology in the form of a frequency-selective surface (FSS) or resonant structure whose electromagnetic resonance characteristics shift measurably in response to changes in the dielectric environment caused by the liquid inside the container. The resonant structure of the Passive Certification Element 626 may be designed such that the resonant frequency, bandwidth, or amplitude of the backscattered signal encodes information about one or more properties of the liquid including permittivity, conductivity, or loss tangent. The design of the resonant structure may be optimized using the Digital Twin Simulation 530 to simulate the interaction between the interrogation signal, the passive element, the container wall, and the enclosed liquid, enabling virtual prototyping of passive certification element designs for different container materials and liquid types without physical fabrication.

With continued reference to FIG. 8, a Power Source 802 provides power to the Monitoring System 808 and other electrical components of the Sump Pump Monitoring System 800. The Power Source 802 may include both AC power and battery backup power to ensure continuous operation during power outages. The AC power connection of the Power Source 802 may provide primary power during normal operation, while the battery backup power may automatically engage when AC power is interrupted, maintaining monitoring and pump control capabilities during storm events when power outages frequently coincide with elevated flooding risk. The dual power configuration of the Power Source 802 may correspond to the power management capabilities described with reference to the Power Management Module 702, where the battery backup provides operational continuity when primary power is unavailable.

A sump basin may contain several internal components that work together with the Monitoring System 808 to manage water accumulation and prevent flooding. A Submersible Pump 806 may be positioned within the sump basin and provides the pumping capability to remove accumulated water from the basin. In alternative embodiments, a pedestal pump may be used. The Submersible Pump 806 may receive activation commands from the Monitoring System 808 based on fluid level measurements and predictive control algorithms executed by the Edge Computing Module 712.

A Float Switch 804 may be positioned within the sump basin and may provide a traditional mechanical sensing mechanism that operates in conjunction with the noninvasive sensing capabilities of the Monitoring System 808. The Float Switch 804 may serve as a backup sensor in a dual-mode configuration as described with reference to the noninvasive Sensor and Float Switch Redundancy Automatic Failover 632, where the Monitoring System 808 acts as the primary controller while also monitoring the state of the Float Switch 804 to detect mechanical failures. In certain embodiments, if the Float Switch 804 fails to actuate when rising water is detected by the Monitoring System 808, the system alerts the user to the mechanical failure while continuing to operate the Submersible Pump 806 correctly based on the noninvasive sensor measurements.

As further shown in FIG. 8, a Pump Stand 810 within the sump basin provides a mounting platform that elevates the Submersible Pump 806 above the bottom of the basin. The Pump Stand 810 may prevent the Submersible Pump 806 from drawing in sediment or debris that accumulates at the bottom of the sump basin. An Inlet 812 provides a pathway for water to enter the sump basin from surrounding soil or drainage systems. An Outlet 824 provides a pathway for water pumped by the Submersible Pump 806 to exit the sump basin and be discharged away from the structure being protected. A Valve 816 may be positioned along the discharge pathway to control water flow and prevent backflow into the sump basin when the Submersible Pump 806 is not operating.

With continued reference to FIG. 8, the Monitoring System 808 connects wirelessly via a Network 818 to a Remote Computer 826 running cloud analytics. The Network 818 may comprise one or more of Wi-Fi, cellular, or other wireless communication technologies supported by the Communication Module 716 within the Monitoring System 808. The Remote Computer 826 may correspond to the Cloud Analytics Platform 528 described with reference to the Edge-To-Cloud Analytics 504 and may provide fleet-wide visibility, analytics dashboards, and centralized management capabilities for distributed sump pump monitoring deployments.

The Remote Computer 826 receives forecast data from a Weather Data Source 820 that provides meteorological information including precipitation forecasts for the geographic location of the Sump Pump Monitoring System 800. The Weather Data Source 820 may comprise one or more of weather service APIs, meteorological data feeds, or other external data sources that provide rainfall predictions. The integration of weather forecast data from the Weather Data Source 820 may enable the Weather-Integrated Pump Activation 582 capability described with reference to the Sump Factors 516 vertical.

In certain embodiments, the predictive hysteresis model executed by the Edge Computing Module 712 or the Remote Computer 826 proactively activates the Submersible Pump 806 in anticipation of predicted rainfall exceeding a threshold even if the current fluid level within the sump basin is below a standard activation threshold. The proactive pump activation may lower the water level in the sump basin before the predicted rainfall event begins, increasing the basin's capacity to handle the anticipated inflow and preventing the Submersible Pump 806 from being overwhelmed during storm events. The predictive hysteresis model may incorporate the current fluid level measured by the Monitoring System 808, the rate of change of the fluid level calculated from historical measurements, and the precipitation forecast data from the Weather Data Source 820 to determine optimal pump activation timing as described with reference to the ML-Based Predictive Hysteresis Control 580.

The Remote Computer 826 sends alerts and reports to a User Device 828 such as a smartphone, tablet, or other mobile computing device. The User Device 828 may receive one or more of status updates, maintenance notifications, or emergency alerts generated by the Multi-Tiered Alert System 610 based on conditions detected by the Monitoring System 808. In certain embodiments, the User Device 828 receives Critical Interventions 644 alerts when the Monitoring System 808 detects conditions indicating imminent flooding risk, sensor failure, or pump malfunction, enabling the user to take immediate action or arrange for emergency service.

In some aspects, the Sump Pump Monitoring System 800 may be integrated with the Parametric Insurance 476 capabilities of the InsurTech Module 405 to provide parametric flood insurance. A parametric flood insurance smart contract deployed on the Immutable Ledger 456 may define trigger conditions based on sensor data from the Monitoring System 808, including one or more of a fluid level exceeding a predefined flood threshold, a rate of fluid level rise exceeding a predefined rate threshold, detection of pump failure via the Float Sensor Redundancy State Diagram 1400, or transition to the Emergency State 1410 where both the noninvasive sensor and the Float Switch 804 have failed. When a trigger condition is met, the Smart Contracts 460 may automatically execute an insurance payout to the property owner without requiring a traditional claims adjustment process, where the blockchain-recorded sensor data from the Monitoring System 808 provides the verified evidentiary basis for the payout. In some aspects, the Dynamic Risk Scoring 478 may continuously adjust the flood insurance premium based on real-time sensor data from the Monitoring System 808, including one or more of the current fluid level, the rate of change of the fluid level, the operational status of the Submersible Pump 806, and the predicted precipitation from the Weather Data Source 820. In some aspects, the Monitoring System 808 may record on the Immutable Ledger 456 a verifiable automated response record comprising the sensor data that triggered a pump activation, the machine learning prediction generated by the Edge Computing Module 712, the pump control action taken (including one or more of the activation timestamp, pump run duration, and volume of water removed), and the state of the Float Switch 804 at the time of activation. The verifiable automated response record may create an immutable, auditable chain linking the detected condition, the system's prediction, and the physical response action, enabling insurers to verify that the automated control system performed as designed and enabling property owners to demonstrate system performance in support of insurance claims, warranty disputes, or regulatory audits.

In some aspects, the Sump Pump Monitoring System 800 may be integrated with the Sustainability Credits Module 406 to generate verifiable environmental credits based on flood prevention outcomes. The Monitoring System 808 may record on the Immutable Ledger 456 verified data regarding the volume of water managed by the Submersible Pump 806 over a defined period, the number of flood prevention events where proactive pump activation by the Weather-Integrated Pump Activation 582 prevented water from exceeding a flood threshold, and the reduction in water damage relative to a baseline established for properties without noninvasive monitoring. In some aspects, the verified flood prevention data recorded on the Immutable Ledger 456 may be used to generate tradeable water management credits representing the quantified environmental benefit of preventing flood events, including one or more of reduced stormwater runoff into municipal systems, reduced contamination of groundwater from flood-borne pollutants, or reduced consumption of materials and energy associated with flood damage repair. The water management credits may be generated when cumulative flood prevention metrics recorded on the Immutable Ledger 456 exceed predefined thresholds, and the credits may be tradeable on environmental credit marketplaces in a manner analogous to the Angel's Share Loss Credits 496 and the Maturation Credits 497 described with reference to the Sustainability Credits Module 406.

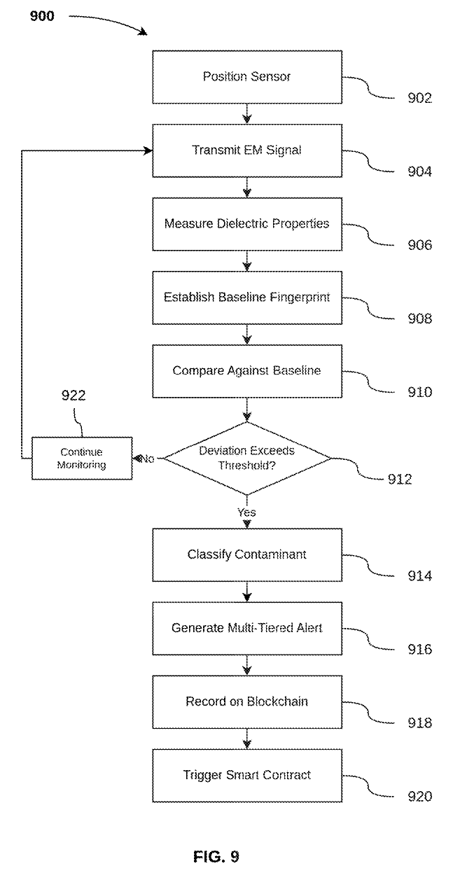

Referring to FIG. 9, a Dielectric Fingerprinting Contamination Detection Method 900 is illustrated as a flowchart depicting a multi-step process for detecting contamination in a liquid within a container using dielectric fingerprinting techniques. The Dielectric Fingerprinting Contamination Detection Method 900 may utilize the dielectric sensing capabilities described with reference to the Dielectric Sensor Array 522 and the Dielectric Sensor Array 708 to identify contamination, adulteration, or degradation of liquids without physical contact with the liquid or penetration of the container wall.

The Dielectric Fingerprinting Contamination Detection Method 900 begins with a Block 902 of positioning the sensor externally adjacent to a container wall. In Block 902, a sensor comprising one or more of the Fixed Sensor Node 622, the Handheld Inspection Device 624, or the Modular Sensor Node Hardware Block Diagram 700 may be positioned on an external surface of the container such that the sensor can interrogate the container contents through the container wall without physical contact with the liquid inside. The external positioning performed in Block 902 may preserve container integrity by avoiding penetration of the container wall, which may be particularly relevant for sealed containers such as aging barrels, sterile pharmaceutical vessels, or fuel tanks where maintaining a hermetic seal is desirable.

With continued reference to FIG. 9, the Dielectric Fingerprinting Contamination Detection Method 900 proceeds to a Block 904 of transmitting a multi-frequency electromagnetic interrogation signal through the container wall. In Block 904, the Dielectric Sensor Array 708 or the Dielectric Sensor Array 522 may transmit electromagnetic signals at multiple frequencies across a predefined spectrum, where the electromagnetic signals propagate through the container wall and interact with the liquid inside the container. The multi-frequency interrogation performed in Block 904 may correspond to the Multi-Frequency Dielectric Spectrum Construction 630 capability described with reference to the Edge Intelligence 606, where interrogating the liquid at multiple frequencies enables construction of a complete dielectric spectrum that serves as a unique fingerprint for the liquid.

A Block 906 measures the dielectric properties of the liquid based on the electromagnetic signals transmitted in Block 904. In Block 906, the sensor may measure one or more of the complex permittivity, the dielectric constant, or the loss tangent of the liquid at each of the multiple frequencies transmitted in Block 904. The dielectric property measurements obtained in Block 906 may capture the frequency-dependent electromagnetic response of the liquid, which varies based on the molecular composition and structure of the liquid. The measurement data generated in Block 906 may be processed by the Edge Computing Module 712 or the Edge Computing Module 524 to construct a dielectric spectrum representing the measured dielectric properties across the interrogation frequency range.

A Block 908 establishes or retrieves a baseline dielectric fingerprint for the liquid. In Block 908, a baseline dielectric fingerprint representing the expected dielectric spectrum of the pure, unadulterated liquid may be established through a direct measurement of a known- good sample or retrieved from a library of baseline fingerprints stored in one or more of the Edge Computing Module 712, the Cloud Analytics Platform 528, or the Digital Twin Simulation 530. The baseline dielectric fingerprint established or retrieved in Block 908 may serve as a reference against which subsequent measurements are compared to detect deviations indicative of contamination, adulteration, or degradation. In certain embodiments, the baseline dielectric fingerprint is generated using synthetic data from the Digital Twin Simulation Environment 440, where the digital twin models the expected dielectric response of the pure liquid based on the liquid's known chemical composition and physical properties.

As further shown in FIG. 9, a Block 910 compares the measured dielectric properties from Block 906 against the baseline dielectric fingerprint from Block 908 using a machine learning model. In Block 910, the Compressed ML Models 628 executed by the Edge Computing Module 712 may analyze the measured dielectric spectrum and compare the measured spectrum to the baseline fingerprint to identify deviations that may indicate contamination. The machine learning model used in Block 910 may be trained on synthetic data generated by the Digital Twin Simulation 530 that includes examples of both clean and contaminated liquid states, enabling the model to recognize patterns in the dielectric spectrum that correspond to various contamination types and concentrations.

A Decision 912 determines whether the deviation between the measured dielectric properties and the baseline fingerprint exceeds a predefined threshold. At Decision 912, the machine learning model may calculate a deviation metric representing the magnitude and characteristics of the difference between the measured spectrum and the baseline fingerprint. If the deviation metric does not exceed the predefined threshold, the Dielectric Fingerprinting Contamination Detection Method 900 proceeds to a Block 922 of continuing periodic monitoring. In Block 922, the system may return to Block 904 to perform another measurement cycle after a predetermined time interval, thereby providing continuous monitoring of the liquid state over time. The periodic monitoring performed in Block 922 may enable detection of gradual contamination events that develop over extended time periods, such as slow water ingress into fuel storage tanks or gradual degradation of pharmaceutical formulations.

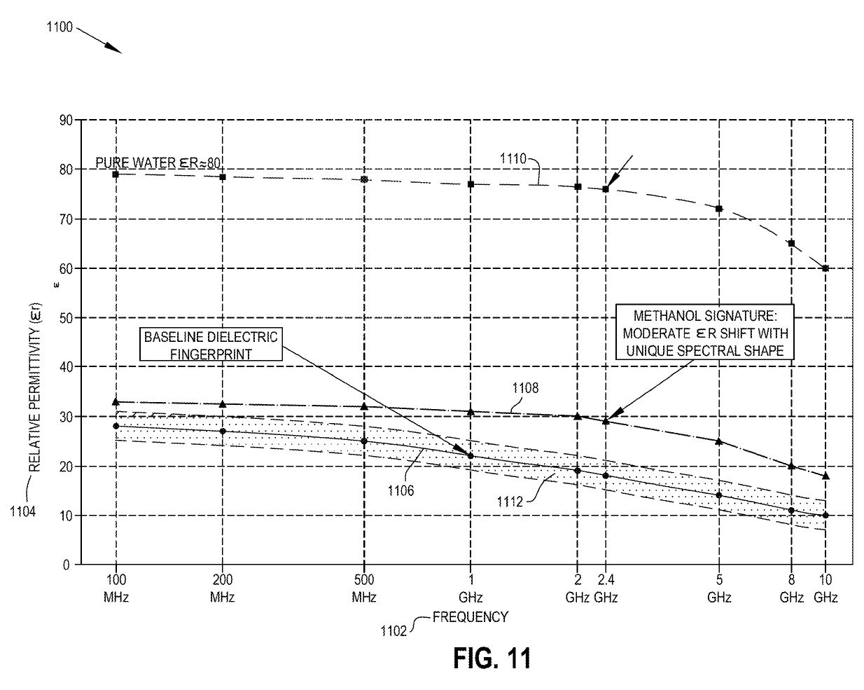

With continued reference to FIG. 9, if the deviation metric at Decision 912 exceeds the predefined threshold, the Dielectric Fingerprinting Contamination Detection Method 900 proceeds to a Block 914 of classifying the contaminant type using a trained machine learning model. In Block 914, the machine learning model may analyze the specific shape and magnitude of the spectral deviation to identify the type of contaminant present in the liquid. The contaminant classification performed in Block 914 may utilize the principle that different contaminants produce distinct patterns in the dielectric spectrum, as described with reference to the Multi-Frequency Dielectric Fingerprint Comparison of FIG. 11 where water contamination produces a large upward shift in permittivity across the spectrum while methanol contamination produces a more moderate shift with a different spectral shape. In certain embodiments, the machine learning model used in Block 914 may be trained on synthetic data from the Digital Twin Simulation 530 that includes examples of the liquid contaminated with various substances at different concentrations, enabling the model to classify contaminants including one or more of water, methanol, chemical solvents, or biological agents based on the unique spectral signature of each contaminant type.

A Block 916 generates a multi-tiered alert with forensic metadata based on the contamination detection and classification performed in Block 910 and Block 914. In Block 916, the Multi-Tiered Alert System 610 may generate an alert at an appropriate severity level based on the type and concentration of the detected contaminant, ranging from Informational Notifications 640 for minor deviations to Critical Interventions 644 for contamination levels that pose safety hazards or require immediate remediation. The alert generated in Block 916 may include the Forensic Metadata for Regulators Compliance and Audit 646, comprising one or more of the sensor data that triggered the alert, the machine learning model prediction, a confidence score indicating the model's certainty in the contaminant classification, or a timestamp indicating when the contamination was detected. The forensic metadata included in the alert may provide a complete record of the contamination event suitable for regulatory compliance submissions, insurance claims, or audit documentation.

A Block 918 records the contamination event immutably on a distributed blockchain. In Block 918, the contamination detection data, the contaminant classification, and the associated forensic metadata may be recorded on the Immutable Ledger 456 of the Blockchain Layer 403 via the Blockchain Permanent Record 638. The blockchain recording performed in Block 918 may create a permanent, tamper-proof record of the contamination event that can be independently verified by authorized parties including one or more of producers, logistics providers, insurers, or regulators. The cryptographic hash of the sensor data may be generated by the Hardware Security Module 714 at the point of data acquisition and recorded on the Immutable Ledger 456, ensuring that the recorded data is resistant to alteration without detection.

A Block 920 triggers a smart contract to execute an automated insurance claim if a parametric insurance policy is active. In Block 920, the Smart Contracts 460 of the Blockchain Layer 403 may automatically execute a payout to a beneficiary if the contamination event recorded in Block 918 meets the trigger conditions defined in a Parametric Insurance 476 policy. The automated claim execution performed in Block 920 may eliminate the delays and disputes associated with traditional claims adjustment processes by automatically verifying that the trigger condition has been met based on the blockchain-recorded sensor data and executing the payout without human intervention. In certain embodiments, the payout executed in Block 920 is denominated in a stablecoin cryptocurrency, enabling instant settlement without currency conversion delays or intermediary processing.

Block 924 completes the contamination detection and response process flow of the Dielectric Fingerprinting Contamination Detection Method 900. Following Block 920, the Dielectric Fingerprinting Contamination Detection Method 900 may return to Block 922 to continue periodic monitoring of the container, enabling detection of additional contamination events or verification that remediation actions have restored the liquid to an acceptable state. The continuous monitoring loop between Block 922 and Block 904 may provide ongoing surveillance of container contents throughout the container's lifecycle, from production through storage and distribution to point of use.

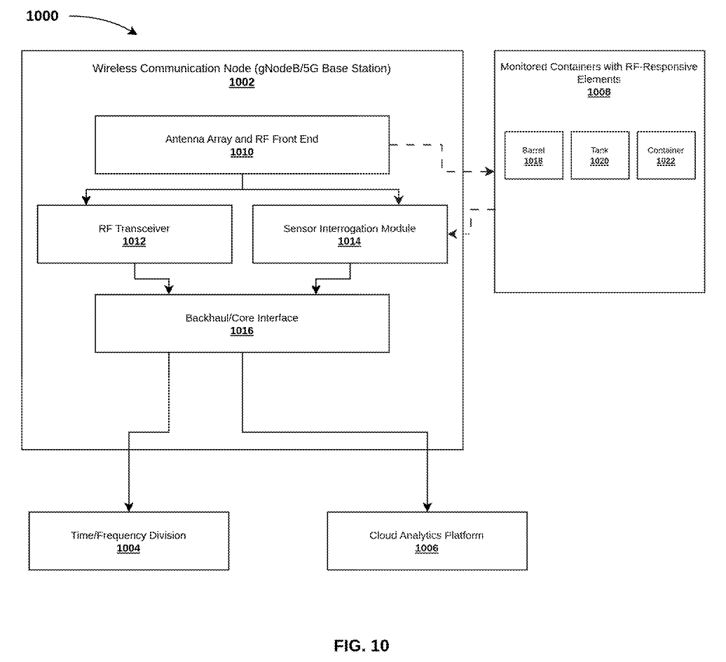

Referring to FIG. 10, a 5G Dual-Use Communication and Sensing Architecture 1000 is illustrated as an architecture diagram depicting the integration of container monitoring capabilities with existing cellular network infrastructure. The 5G Dual-Use Communication and Sensing Architecture 1000 may enable the platform to leverage wireless communication nodes that are deployed for conventional cellular communication services to perform container monitoring functions, thereby reducing infrastructure deployment costs and utilizing existing network coverage for wide-area sensing applications.

A Wireless Communication Node 1002 forms the central component of the 5G Dual-Use Communication and Sensing Architecture 1000 and may serve as both a cellular base station providing conventional communication services and a sensor interrogator for monitoring containers equipped with RF-responsive elements. The Wireless Communication Node 1002 may comprise a gNodeB base station or other cellular network node that provides dual-use functionality by dynamically allocating resources between communication and sensing operations. The dual-use configuration of the Wireless Communication Node 1002 may correspond to the 5G/Cellular Infrastructure Integration 602 described with reference to the Advanced Deployment and Infrastructure Architecture 600, where the 5G Base Station Wireless Communication Node 616 provides both conventional cellular and sensor interrogation capabilities.

With continued reference to FIG. 10, an Antenna Array and RF Front End 1010 within the Wireless Communication Node 1002 may direct beamformed RF signals towards monitored containers that are equipped with RF-responsive sensor elements. The Antenna Array and RF Front End 1010 may comprise one or more of phased array antennas, multiple- input multiple-output (MIMO) antenna configurations, or other antenna systems capable of directing electromagnetic energy towards specific spatial locations. The beamforming capabilities of the Antenna Array and RF Front End 1010 may enable the Wireless Communication Node 1002 to focus interrogation signals on individual containers or groups of containers within the coverage area, improving signal-to-noise ratio and enabling characterization of container contents based on the reflected signals received by Sensor Interrogation Module 1014 from the Monitored Containers 1008. In certain embodiments, the Antenna Array and RF Front End 1010 utilizes the same antenna elements for both conventional cellular communication and sensor interrogation, with the beamforming configuration being adjusted based on whether the system is operating in communication mode or sensing mode.

An RF Transceiver 1012 within the Wireless Communication Node 1002 handles conventional cellular communication during designated communication time slots. The RF Transceiver 1012 may perform one or more of signal modulation, demodulation, frequency conversion, or amplification functions for cellular communication services including one or more of voice, data, or messaging services provided to mobile devices within the coverage area of the Wireless Communication Node 1002. The RF Transceiver 1012 may operate according to 5G New Radio (NR) specifications or other cellular communication standards, providing the conventional base station functionality that enables the Wireless Communication Node 1002 to serve as part of a cellular network infrastructure.

A Sensor Interrogation Module 1014 within the Wireless Communication Node 1002 uses the Antenna Array and RF Front End 1010 to send interrogation signals and analyze reflected signals to characterize container contents during sensing time slots. The Sensor Interrogation Module 1014 may transmit interrogation signals towards containers equipped with RF-responsive elements and analyze the backscattered signals to determine properties of the container contents based on the electromagnetic interaction between the interrogation signals and the container contents. The Sensor Interrogation Module 1014 may correspond to the Backscatter-Based Sensing of Passive RF-Response Elements 620 capability described with reference to the 5G/Cellular Infrastructure Integration 602, where the backscattered signal is modulated by an interaction between a passive element's antenna and the dielectric properties of the liquid inside the container.

As further shown in FIG. 10, a Time/Frequency Division 1004 component within the 5G Dual-Use Communication and Sensing Architecture 1000 provides dynamic allocation of time slots between communication and sensing operations. The Time/Frequency Division 1004 may interleave communication time slots with sensing time slots such that the Wireless Communication Node 1002 handles conventional cellular communication via the RF Transceiver 1012 during designated communication time slots and performs sensor interrogation via the Sensor Interrogation Module 1014 during interleaved sensing time slots. In certain embodiments, the Time/Frequency Division 1004 allocates time slots based on one or more of communication traffic demand, sensing priority requirements, or scheduling algorithms that balance communication quality of service with sensing update rates. The dynamic allocation performed by the Time/Frequency Division 1004 may enable the Wireless Communication Node 1002 to provide both communication and sensing services without dedicated hardware for each function, thereby reducing infrastructure costs while maintaining service quality for both functions.

A Backhaul/Core Interface 1016 within the Wireless Communication Node 1002 provides connectivity to backend infrastructure for both communication and sensing data. The Backhaul/Core Interface 1016 may transmit conventional cellular traffic to the mobile network core for routing and processing according to standard cellular network protocols. The Backhaul/Core Interface 1016 may also transmit sensor data collected by the Sensor Interrogation Module 1014 to a Cloud Analytics Platform 1006 for aggregation, model training, and alerting. The Cloud Analytics Platform 1006 may correspond to the Cloud Analytics Platform 528 described with reference to the Edge-To-Cloud Analytics 504 and may provide fleet-wide visibility, analytics dashboards, and centralized management capabilities for the distributed container monitoring network enabled by the 5G Dual-Use Communication and Sensing Architecture 1000. The Backhaul/Core Interface 1016 may receive user data from RF Transceiver 1012 and sensor data from Sensor Interrogate Module 1014. The Backhaul/Core Interface 1016 may send aggregated data to Cloud Analytics Platform 1006.

With continued reference to FIG. 10, Monitored Containers 1008 represent the containers that are monitored by the Wireless Communication Node 1002 using the sensor interrogation capabilities of the 5G Dual-Use Communication and Sensing Architecture 1000. The Monitored Containers 1008 may comprise containers of various types that have been equipped with passive or semi-passive RF-responsive elements that interact with the interrogation signals transmitted by the Sensor Interrogation Module 1014. The RF-responsive elements affixed to the Monitored Containers 1008 may correspond to the Passive Certification Element 626 described with reference to the Device Form Factors 604, where the passive element has an electromagnetic response that changes based on a property of a liquid inside the container.

The Monitored Containers 1008 may include any suitable one or more containers. For example, and without limitation, the Monitored Containers 1008 may include one or more of a Barrel 1018, a Tank 1020, or a Container 1022.

In certain embodiments, the 5G Dual-Use Communication and Sensing Architecture 1000 enables wide-area monitoring of distributed container assets without requiring dedicated sensor infrastructure at each container location. The Wireless Communication Node 1002 may interrogate multiple containers within the coverage area during each sensing time slot allocated by the Time/Frequency Division 1004, with the beamforming capabilities of the Antenna Array and RF Front End 1010 enabling sequential or parallel interrogation of containers at different locations. The sensor data collected from the Barrel 1018, the Tank 1020, the Container 1022, and/or other monitored containers may be transmitted via the Backhaul/Core Interface 1016 to the Cloud Analytics Platform 1006 for processing, where machine learning models may analyze the backscatter characteristics to determine container content properties and detect anomalies indicative of one or more of contamination, leakage, or theft. The Cloud Analytics Platform 1006 may record sensor data and predictions on the Immutable Ledger 456 via the Blockchain Permanent Record 638, creating a tamper-proof audit trail for the monitored containers that supports one or more of regulatory compliance, insurance applications, or supply chain verification.

Referring to FIG. 11, a Multi-Frequency Dielectric Fingerprint Comparison 1100 is illustrated as a graph depicting the relationship between electromagnetic interrogation frequency and the dielectric response of liquids in various states of purity and contamination. The Multi-Frequency Dielectric Fingerprint Comparison 1100 demonstrates the principle underlying the contamination detection capabilities described with reference to the Dielectric Fingerprinting Contamination Detection Method 900, where different substances produce distinguishable dielectric fingerprints that enable identification of contamination type based on spectral characteristics.

A Frequency Axis 1102 defines the horizontal dimension of the plot space in the Multi-Frequency Dielectric Fingerprint Comparison 1100 and represents the range of electromagnetic interrogation frequencies used to construct the dielectric spectrum. A Relative Permittivity Axis 1104 defines the vertical dimension of the plot space and represents the measured dielectric constant of the liquid at each interrogation frequency. The Relative Permittivity Axis 1104 indicates the degree to which the liquid polarizes in response to an applied electromagnetic field, where higher permittivity values indicate greater polarization response.

With continued reference to FIG. 11, a Clean Bourbon Line 1106 represents the baseline dielectric fingerprint for pure, unadulterated bourbon and shows a gradual decrease in permittivity as frequency increases along the Frequency Axis 1102. The Clean Bourbon Line 1106 may correspond to the baseline dielectric fingerprint established or retrieved in Block 908 of the Dielectric Fingerprinting Contamination Detection Method 900, against which subsequent measurements are compared to detect deviations indicative of contamination. The gradual decrease in permittivity exhibited by the Clean Bourbon Line 1106 may reflect the frequency-dependent dielectric response characteristic of the alcohol-water mixture that constitutes bourbon, where the molecular relaxation processes that contribute to polarization become less effective at higher frequencies.

A Water-Contaminated Line 1110 in the Multi-Frequency Dielectric Fingerprint Comparison 1100 shows the effect of water contamination on the dielectric spectrum of bourbon. The Water-Contaminated Line 1110 exhibits an upward shift in permittivity across the entire spectrum relative to the Clean Bourbon Line 1106. The upward shift exhibited by the Water-Contaminated Line 1110 may result from the high permittivity of water, which has a relative permittivity of approximately 80 at room temperature compared to lower permittivity values for alcohols. The Water-Contaminated Line 1110 may demonstrate that water contamination produces a large, broad increase in permittivity that is readily distinguishable from the baseline represented by the Clean Bourbon Line 1106.

A Methanol-Contaminated Line 1108 in the Multi-Frequency Dielectric Fingerprint Comparison 1100 shows the effect of methanol contamination on the dielectric spectrum of bourbon. The Methanol-Contaminated Line 1108 exhibits a more moderate upward shift relative to the Clean Bourbon Line 1106 compared to the more dramatic shift exhibited by the Water-Contaminated Line 1110. The more moderate shift exhibited by the Methanol-Contaminated Line 1108 may result from methanol having a permittivity closer to the baseline bourbon mixture than water. The Methanol-Contaminated Line 1108 may exhibit a unique spectral shape that differs from both the Clean Bourbon Line 1106 and the Water- Contaminated Line 1110, where the frequency-dependent characteristics of the methanol contamination produce a distinctive pattern that enables the machine learning models described with reference to Block 914 of the Dielectric Fingerprinting Contamination Detection Method 900 to distinguish methanol contamination from water contamination based on the spectral signature.

As further shown in FIG. 11, a Detection Threshold 1112 region surrounds the Clean Bourbon Line 1106 and represents the acceptable range of variation around the baseline within which measured dielectric properties are considered consistent with uncontaminated liquid. The Detection Threshold 1112 may correspond to the predefined threshold evaluated at Decision 912 of the Dielectric Fingerprinting Contamination Detection Method 900, where deviations exceeding the Detection Threshold 1112 trigger contamination classification and alerting procedures. The Detection Threshold 1112 may account for one or more of measurement noise, temperature variations, or minor compositional variations that do not indicate contamination, thereby reducing false positive detections while maintaining sensitivity to genuine contamination events.

The Multi-Frequency Dielectric Fingerprint Comparison 1100 demonstrates that by analyzing the dielectric response across multiple frequencies along the Frequency Axis 1102, the system can not only detect a deviation from the baseline represented by the Clean Bourbon Line 1106 but also classify the specific type of contaminant based on the unique shape and magnitude of the spectral shift. The Water-Contaminated Line 1110 and the Methanol- Contaminated Line 1108 may exhibit distinguishable spectral characteristics that enable the Compressed ML Models 628 executed by the Edge Computing Module 712 to identify whether detected contamination comprises water, methanol, or other substances based on the pattern of deviation from the Clean Bourbon Line 1106. In certain embodiments, the machine learning models may be trained on synthetic data from the Digital Twin Simulation 530 that includes examples of the liquid contaminated with various substances at different concentrations, enabling classification of contaminants based on the unique spectral signatures illustrated by the Multi-Frequency Dielectric Fingerprint Comparison 1100.

FIG. 11 provides examples of a Water-Contaminated Line 1110, Clean Bourbon Line 1106, and Methanol-Contaminated Line 1108 by way of illustration rather than limitation. Any one or more suitable fluids may be evaluated using the technology described herein.

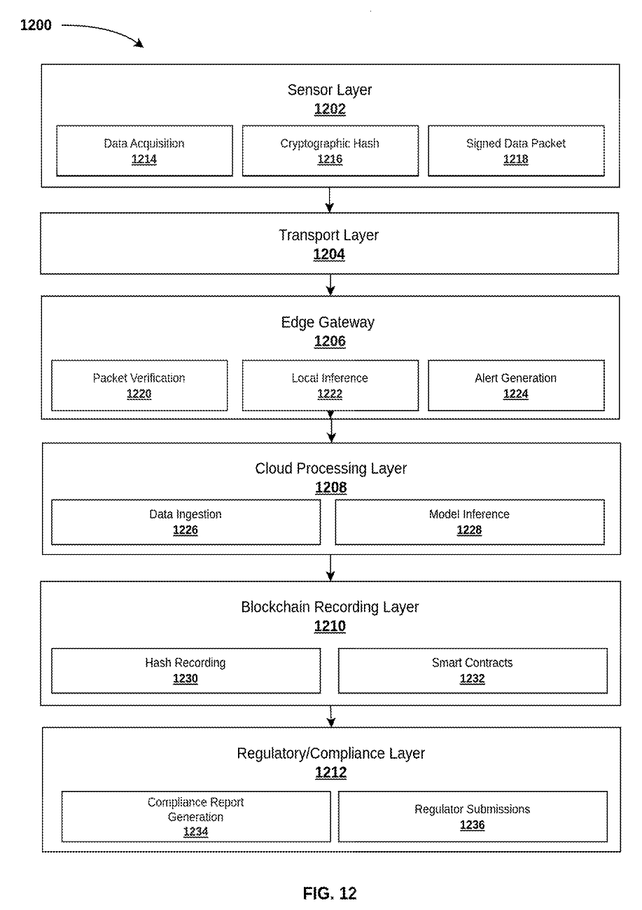

Referring to FIG. 12, an Integrity Monitoring Data Pipeline Architecture 1200 is illustrated as a layered architecture diagram depicting the data flow from sensor acquisition through regulatory submission, organized into six sequential layers that process, secure, and record container monitoring data. The Integrity Monitoring Data Pipeline Architecture 1200 may provide a comprehensive framework for ensuring data integrity, enabling automated processing, and supporting regulatory compliance throughout the data lifecycle from the point of sensor measurement to final submission to regulatory authorities. A Sensor Layer 1202 forms the uppermost layer of the Integrity Monitoring Data Pipeline Architecture 1200 and contains the components responsible for capturing, securing, and packaging sensor readings at the point of data acquisition.

The Sensor Layer 1202 may correspond to the data acquisition and security functions performed by the Modular Sensor Node Hardware Block Diagram 700, where sensor data is collected and cryptographically secured before transmission to downstream processing components.

With continued reference to FIG. 12, a Data Acquisition 1214 component within the Sensor Layer 1202 captures raw sensor readings from the sensing components of the monitoring system. The Data Acquisition 1214 component may receive data from one or more of the FMCW Radar Module 706, the Dielectric Sensor Array 708, or the Auxiliary Sensor Ports 704, collecting the measurements that form the basis for container content characterization and integrity monitoring. The Data Acquisition 1214 component may perform initial signal conditioning and digitization of analog sensor outputs to produce digital data suitable for subsequent processing and transmission.

A Cryptographic Hash 1216 component within the Sensor Layer 1202 generates a cryptographic hash of the sensor data captured by the Data Acquisition 1214 at the moment of data creation. The Cryptographic Hash 1216 may be performed by the Hardware Security Module 714 within the sensor node, ensuring that the hash is generated within a secure, tamper- resistant environment before the data leaves the sensor node. The cryptographic hash generated by the Cryptographic Hash 1216 may serve as a digital fingerprint of the original sensor data that enables verification of data integrity at any subsequent point in the data lifecycle, where any modification to the original data would result in a different hash value and thereby enable detection of tampering.

A Signed Data Packet 1218 component within the Sensor Layer 1202 packages the sensor data and the cryptographic hash into a signed data packet for transmission to downstream processing components. The Signed Data Packet 1218 may include one or more of the raw sensor data, the cryptographic hash generated by the Cryptographic Hash 1216, a digital signature generated by the Hardware Security Module 714, a timestamp indicating when the data was acquired, or a sensor identifier indicating which sensor node generated the data. The digital signature included in the Signed Data Packet 1218 may enable verification of the data source, ensuring that the data originated from an authorized sensor node and has not been modified since signing.

As further shown in FIG. 12, a Transport Layer 1204 handles the transmission of signed data packets from the Sensor Layer 1202 to downstream processing components. The Transport Layer 1204 may utilize the TLS Encryption 634 described with reference to the End- To-End Security Architecture 608 to protect data during transmission, establishing encrypted communication channels between the sensor node and receiving infrastructure. The Transport Layer 1204 may utilize the Communication Module 716 to transmit the Signed Data Packet 1218 via one or more of Bluetooth Low Energy, Wi-Fi, LoRaWAN, or 5G cellular connectivity based on the deployment environment and connectivity requirements.

An Edge Gateway 1206 receives and processes the signed data packets transmitted via the Transport Layer 1204, providing local processing capabilities that reduce latency and enable operation in environments with limited or intermittent cloud connectivity. The Edge Gateway 1206 may correspond to the Edge Intelligence 606 capabilities described with reference to the Advanced Deployment and Infrastructure Architecture 600, where intelligent processing is performed at or near the point of data collection.

With continued reference to FIG. 12, a Packet Verification 1220 component within the Edge Gateway 1206 verifies the integrity and authenticity of received data packets. The Packet Verification 1220 may verify the digital signature included in the Signed Data Packet 1218 to confirm that the data originated from an authorized sensor node. The Packet Verification 1220 component may also verify the cryptographic hash to confirm that the sensor data has not been modified during transmission. Data packets that fail verification may be rejected or flagged for investigation, ensuring that only authentic, unmodified data proceeds to subsequent processing stages.

A Local Inference 1222 component within the Edge Gateway 1206 executes machine learning models to generate predictions based on the verified sensor data. The Local Inference 1222 may execute the Compressed ML Models 628 to perform real-time inference without requiring cloud connectivity, enabling immediate generation of predictions regarding one or more of container fill level, content composition, or contamination status. The Local Inference 1222 may correspond to the local inference capabilities provided by the Edge Computing Module 524 or the Edge Computing Module 712, where predictions are generated at the point of data collection to reduce latency and bandwidth requirements.

An Alert Generation 1224 component within the Edge Gateway 1206 generates alerts based on the predictions produced by the Local Inference 1222. The Alert Generation 1224 component may implement the Multi-Tiered Alert System 610, generating one or more of Informational Notifications 640, Warning Alerts 642, or Critical Interventions 644 based on the severity of detected conditions. The Alert Generation 1224 may include the Forensic Metadata for Regulators Compliance and Audit 646 with each generated alert, providing a complete record of the sensor data, prediction, confidence score, and timestamp associated with the alert condition.

As further shown in FIG. 12, a Cloud Processing Layer 1208 provides centralized processing capabilities for data aggregation, advanced analytics, and model management across the distributed sensor network. The Cloud Processing Layer 1208 may correspond to the Cloud Analytics Platform 528 described with reference to the Edge-To-Cloud Analytics 504, providing fleet-wide visibility and centralized management capabilities.

A Data Ingestion 1226 component within the Cloud Processing Layer 1208 receives data from the Edge Gateway 1206 and ingests the data into the cloud analytics infrastructure. The Data Ingestion 1226 may aggregate data from multiple sensor nodes across the distributed monitoring network, normalizing data formats and organizing data for subsequent analysis and storage. The Data Ingestion 1226 may validate incoming data against expected schemas and flag anomalous data for review.

A Model Inference 1228 component within the Cloud Processing Layer 1208 executes machine learning models on the aggregated sensor data to generate predictions and analytics at the fleet level. The Model Inference 1228 may execute more computationally intensive models than those deployed to edge devices, leveraging cloud computing resources to perform advanced analytics including one or more of cross-container correlation analysis, fleet-wide trend detection, or predictive maintenance scheduling.

With continued reference to FIG. 12, a Blockchain Recording Layer 1210 provides immutable data recording capabilities that create a tamper-proof audit trail for sensor data, predictions, and automated actions. The Blockchain Recording Layer 1210 may correspond to the Blockchain Layer 403 described with reference to the Blockchain-Enabled Ecosystem Architecture 400, providing the trust infrastructure that enables multi-party verification and supports novel business models.

A Hash Recording 1230 component within the Blockchain Recording Layer 1210 records the cryptographic hashes of sensor data on the Immutable Ledger 456. The Hash Recording 1230 may record the hash generated by the Cryptographic Hash 1216 at the Sensor Layer 1202, creating a permanent, distributed record that enables verification of data integrity at any future time. The Hash Recording 1230 may correspond to the Blockchain Permanent Record 638 described with reference to the End-To-End Security Architecture 608, where the hash is recorded on a distributed ledger that is resistant to alteration without detection.

A Smart Contracts 1232 component within the Blockchain Recording Layer 1210 provides automated execution capabilities that trigger predefined actions when specified conditions are met. The Smart Contracts 1232 may correspond to the Smart Contracts 460 described with reference to the Blockchain Layer 403, enabling automated business processes including one or more of parametric insurance claim execution, collateral valuation updates, or regulatory reporting triggers. In certain embodiments, the Smart Contracts 1232 may execute automated response verification by recording on the blockchain the sensor data that triggered a control action, the AI prediction generated by the machine learning model, and/or the control action taken, thereby creating an immutable, auditable record of automated responses that can be independently verified by authorized parties.

A Regulatory/Compliance Layer 1212 forms the bottom layer of the Integrity Monitoring Data Pipeline Architecture 1200 and provides capabilities for generating compliance documentation and submitting data to regulatory authorities. The Regulatory/Compliance Layer 1212 may leverage the verified, blockchain-recorded data from the Blockchain Recording Layer 1210 to produce documentation that satisfies regulatory requirements across multiple industries.

A Compliance Report Generation 1234 component within the Regulatory/Compliance Layer 1212 automatically generates compliance reports based on the verified sensor data and predictions recorded throughout the Integrity Monitoring Data Pipeline Architecture 1200. The Compliance Report Generation 1234 component may generate reports compliant with one or more of TTB regulations for spirits industry applications, FDA regulations for pharmaceutical applications, or EPA regulations for water quality applications. The Compliance Report Generation 1234 component may format reports according to regulatory specifications and include references to the blockchain-recorded data that supports the reported values, enabling regulators to independently verify the underlying data.

A Regulator Submissions 1236 component within the Regulatory/Compliance Layer 1212 transmits compliance reports and supporting data to regulatory authorities. The Regulator Submissions 1236 may transmit reports generated by the Compliance Report Generation 1234 via secure channels to regulatory databases or submission portals. The Regulator Submissions 1236 may include references to the blockchain-recorded hashes that enable regulators to verify the integrity of submitted data against the immutable records on the Immutable Ledger 456, providing confidence that the submitted data has not been altered since the original sensor measurements were taken.

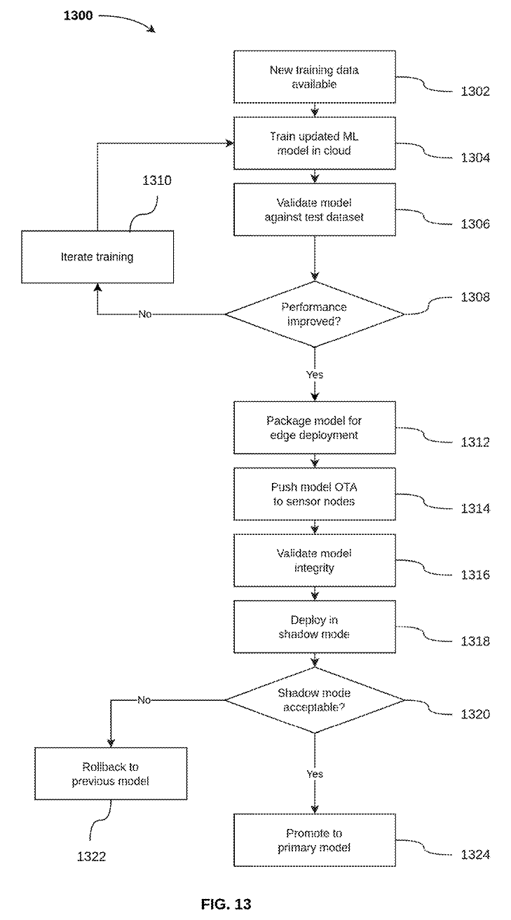

Referring to FIG. 13, an Over-the-Air ML Model Update Method 1300 is illustrated as a flowchart depicting a multi-step lifecycle for updating machine learning models on deployed sensor nodes without requiring physical access to the sensor nodes. The Over-the- Air ML Model Update Method 1300 may provide a systematic process for developing, validating, deploying, and monitoring updated machine learning models that enables continuous improvement of deployed sensor nodes as new training data becomes available and improved models are developed.

The Over-the-Air ML Model Update Method 1300 begins with a Block 1302 of receiving new training data from one or more of the Digital Twin Simulation 530 or field sensor data collection from deployed sensor nodes. In Block 1302, new training data may become available through one or more of synthetic data generation by the Digital Twin Simulation Environment 440, real-world sensor data collected by the Multi-Modal Sensor Array 424, or data identified through the Feedback Loop 432 as representing edge cases or low-confidence predictions that warrant additional model training. The new training data received in Block 1302 may include labeled samples that expand the coverage of the training dataset to include conditions not well-represented in previous training iterations.

With continued reference to FIG. 13, the Over-the-Air ML Model Update Method 1300 proceeds to a Block 1304 of training an updated machine learning model in a cloud environment using the new training data received in Block 1302. In Block 1304, the AI/ML Model Training and Inference 430 component may train an updated model using the Compute Resources 434 provided by the Cloud Orchestration 410. The model training performed in Block 1304 may utilize one or more of deep neural network architectures, transfer learning techniques applied via the Model Adaptation Layer 536, or federated learning approaches coordinated by the Federated Learning 414 module. The updated model trained in Block 1304 may incorporate the new training data to improve prediction accuracy for conditions that were previously underrepresented in the training dataset.

A Block 1306 validates the updated machine learning model against a held-out test dataset that was not used during training. In Block 1306, the updated model trained in Block 1304 may undergo rigorous testing to assess the model's ability to generalize to data not seen during training. The validation performed in Block 1306 may evaluate one or more of prediction accuracy, false positive rate, false negative rate, or inference latency to determine whether the updated model provides improved performance relative to the currently deployed model. The held-out test dataset used in Block 1306 may include both synthetic data from the Digital Twin Simulation 530 and real-world data collected from deployed sensor nodes to assess performance across both simulated and actual operating conditions.

As further shown in FIG. 13, a Decision 1308 determines whether the performance of the updated model has improved over the currently deployed model based on the validation results from Block 1306. At Decision 1308, the validation metrics may be compared against the corresponding metrics for the currently deployed model to determine whether the updated model provides a performance improvement that justifies deployment. If the performance has not improved at Decision 1308, the Over-the-Air ML Model Update Method 1300 proceeds to a Block 1310 of iterating the training process by adjusting hyperparameters and augmenting the training data. In Block 1310, one or more of learning rate, batch size, network architecture, regularization parameters, or data augmentation strategies may be adjusted to improve model performance. The Block 1310 may also augment the training data with additional synthetic samples generated by the Digital Twin Simulation 530 or the Generative AI 412 module to address identified weaknesses in model performance. Following Block 1310, the Over-the-Air ML Model Update Method 1300 returns to Block 1304 to train a new version of the updated model using the adjusted hyperparameters and augmented training data.

If the performance has improved at Decision 1308, the Over-the-Air ML Model Update Method 1300 proceeds to a Block 1312 of packaging the updated model for edge deployment using quantization and compression techniques. In Block 1312, the full-precision model trained in Block 1304 may be converted to a compressed model variant suitable for execution on resource-constrained edge devices. The packaging performed in Block 1312 may apply one or more of weight quantization to reduce numerical precision, pruning to remove redundant network connections, or knowledge distillation to create a smaller model that approximates the behavior of the full-precision model. In certain embodiments, Block 1312 applies 8-bit integer quantization to create a compressed model variant with reduced memory footprint and computational requirements suitable for deployment on the Edge Computing Module 524 or the Edge Computing Module 712. The compressed model variant produced in Block 1312 may correspond to the Compressed ML Models 628 described with reference to the Edge Intelligence 606.

With continued reference to FIG. 13, a Block 1314 pushes the packaged model over-the-air to target sensor nodes via the Communication Module 526 or the Communication Module 716. In Block 1314, the Cloud Analytics Platform 528 may establish a communication link with each target sensor node and transmit the compressed model variant packaged in Block 1312. The over-the-air transmission performed in Block 1314 may utilize one or more of Wi- Fi, LoRaWAN, or 5G cellular connectivity provided by the Communication Module 716 to deliver the updated model to sensor nodes without requiring physical access to the sensor nodes. The Block 1314 may correspond to the Over-the-Air ML Model Updates 650 capability described with reference to the Predictive Analytics 612.

A Block 1316 validates the integrity of the received model at the sensor node via checksum verification and digital signature validation. In Block 1316, the sensor node may compute a checksum of the received model file and compare the computed checksum against an expected checksum transmitted with the model to verify that the model file was not corrupted during transmission. The Block 1316 may also verify a digital signature associated with the model to confirm that the model originated from an authorized source and has not been tampered with. In certain embodiments, the Hardware Security Module 714 performs the digital signature validation in Block 1316 to ensure that signature verification occurs within a secure, tamper-resistant environment. Model files that fail integrity validation in Block 1316 may be rejected, and the sensor node may request retransmission of the model from the Cloud Analytics Platform 528.

A Block 1318 deploys the validated model in shadow mode, where the new model runs in parallel with the currently deployed production model for real-world comparison without affecting live operations. In Block 1318, the Edge Computing Module 712 may execute both the new model received via Block 1314 and the existing production model on incoming sensor data, generating predictions from both models for each measurement cycle. The shadow mode deployment performed in Block 1318 may enable evaluation of the new model's performance on real-world data under actual operating conditions without affecting the predictions, alerts, or control actions generated by the production system. The predictions generated by the new model during shadow mode may be logged and compared against the predictions generated by the production model to assess whether the new model provides improved accuracy, reduced false positive rates, or other performance improvements in the actual deployment environment.

As further shown in FIG. 13, a Decision 1320 determines whether the shadow mode performance of the new model is acceptable based on the comparison data collected during Block 1318. At Decision 1320, the performance metrics of the new model operating in shadow mode may be evaluated against predefined acceptance criteria to determine whether the new model should be promoted to production status. The acceptance criteria evaluated at Decision 1320 may include one or more of prediction accuracy relative to the production model, consistency of predictions across varying operating conditions, or absence of anomalous predictions that would indicate model instability.

If the shadow mode performance is not acceptable at Decision 1320, the Over-the- Air ML Model Update Method 1300 proceeds to a Block 1322 of rolling back to the previous model. In Block 1322, the new model deployed in shadow mode may be automatically discarded, and the existing production model may continue operating without interruption. The rollback performed in Block 1322 may ensure that sensor node operation is not degraded by deployment of a model that performs poorly in the actual deployment environment, even if the model performed well during validation on held-out test data in Block 1306. The automatic rollback mechanism provided by Block 1322 may protect against situations where the domain gap between validation data and real-world operating conditions causes the new model to underperform relative to the existing production model. Following Block 1322, the Over-the- Air ML Model Update Method 1300 may return to Block 1310 to iterate the training process with additional data or adjusted parameters informed by the shadow mode performance analysis.

If the shadow mode performance is acceptable at Decision 1320, the Over-the-Air ML Model Update Method 1300 proceeds to a Block 1324 of promoting the new model to become the primary production model. In Block 1324, the new model that has been validated through both held-out test data evaluation in Block 1306 and shadow mode real-world evaluation in Block 1318 may replace the existing production model as the primary model used for generating predictions, alerts, and control actions. The promotion performed in Block 1324 may update the configuration of the Edge Computing Module 712 to designate the new model as the production model and may archive the previous production model to enable future rollback if issues are subsequently identified.

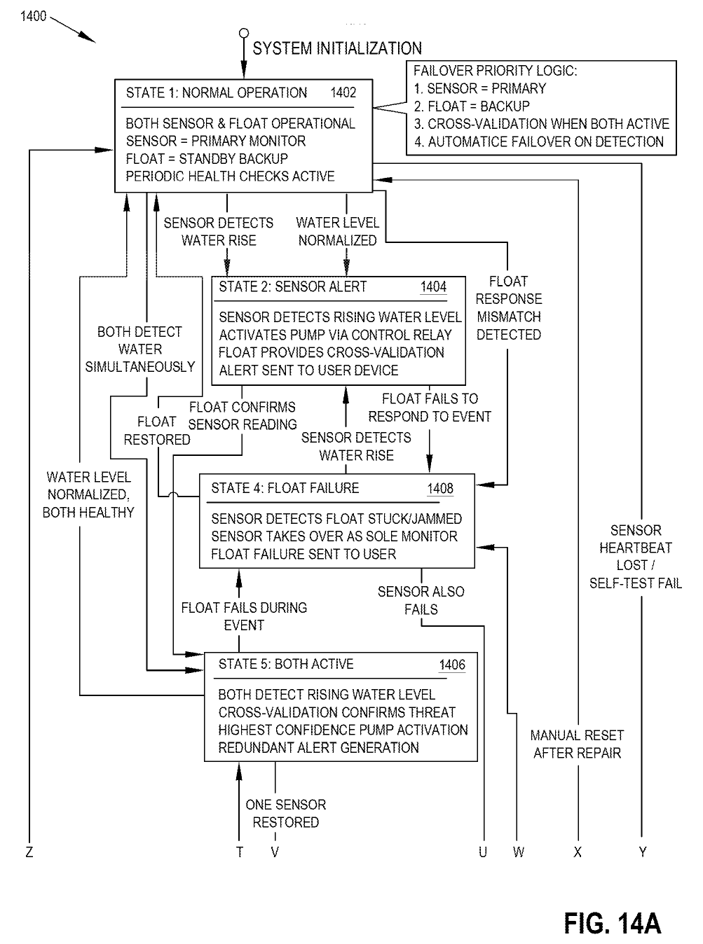

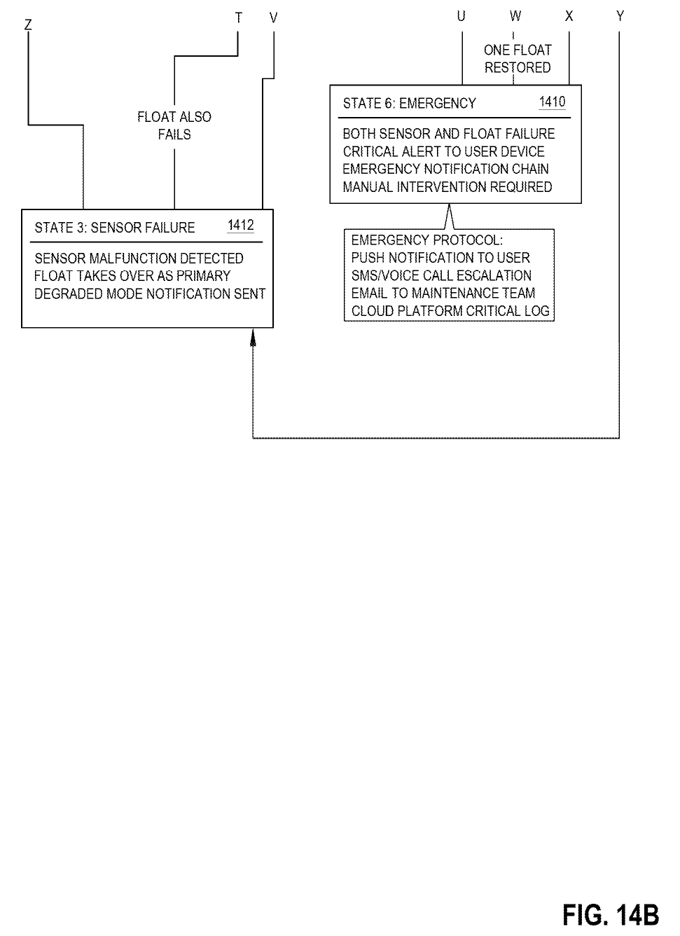

Referring to FIGS. 14A and 14B, a Float Sensor Redundancy State Diagram 1400 is illustrated as a state diagram depicting the dual-mode failover logic between the noninvasive sensor and a traditional float switch for applications requiring maximum reliability. The Float Sensor Redundancy State Diagram 1400 may provide a systematic framework for managing sensor redundancy and failure detection in the Sump Pump Monitoring System 800, ensuring continuous pump control even when one or more sensing components fail. The Float Sensor Redundancy State Diagram 1400 may correspond to the noninvasive Sensor and Float Switch Redundancy Automatic Failover 632 capability described with reference to the Edge Intelligence 606, where the system operates in a dual-mode configuration with automatic detection of failure in the primary sensor and failover to the backup sensor.

A Normal Operation State 1402 forms the initial state of the Float Sensor Redundancy State Diagram 1400 and represents the standard operating condition where both the noninvasive sensor and the Float Switch 804 are functioning correctly. In the Normal Operation State 1402, the Monitoring System 808 may serve as the primary monitor for determining fluid level within the sump basin, while the Float Switch 804 serves as a standby backup that can assume primary monitoring responsibilities if the noninvasive sensor fails. The Normal Operation State 1402 may represent the steady-state condition during periods when the water level within the sump basin is below activation thresholds and both sensing modalities are reporting consistent, expected readings.

With continued reference to FIGS. 14A and 14B, a Sensor Alert State 1404 represents the condition where the noninvasive sensor within the Monitoring System 808 has detected rising water within the sump basin and has activated the Submersible Pump 806 in response. The system may transition from the Normal Operation State 1402 to the Sensor Alert State 1404 when the fluid level measured by the FMCW Radar Module 706 exceeds an activation threshold determined by the ML-Based Predictive Hysteresis Control 580 or the Weather-Integrated Pump Activation 582. In the Sensor Alert State 1404, the Submersible Pump 806 may operate to remove accumulated water from the sump basin, and the Monitoring System 808 may continue monitoring the fluid level to determine when the water level has decreased sufficiently to deactivate the pump. The Sensor Alert State is not limited to occurring only when a rise in water is detected; rather, a Sensor Alert State 1404 may trigger in any condition in which the system determines that the sufficient risk of flooding exists. Any one or more factors, such as water level, rate of change in water level, weather data, or other suitable data may be considered without departing from the scope of the present disclosure. Water is used by way of example rather than limitation; other fluids other than water may be tracked.

A Both Active State 1406 represents the condition where both the noninvasive sensor and the Float Switch 804 have simultaneously detected rising water within the sump basin. The system may transition to the Both Active State 1406 when both sensing modalities independently indicate that the water level has exceeded their respective activation thresholds. The Both Active State 1406 may provide high-confidence, cross-validated activation of the Submersible Pump 806, where the agreement between the noninvasive sensor and the Float Switch 804 increases confidence that the detected condition is genuine rather than a sensor malfunction or environmental artifact. In certain embodiments, the Both Active State 1406 may trigger enhanced logging or notification to indicate that the pump activation has been confirmed by multiple independent sensing modalities.

As further shown in FIGS. 14A and 14B, a Float Failure State 1408 represents the condition where the Float Switch 804 has failed while the noninvasive sensor within the Monitoring System 808 remains operational. The system may transition to the Float Failure State 1408 from one or more of the Sensor Alert State 1404 or the Both Active State 1406 when the Monitoring System 808 detects that the Float Switch 804 has failed to respond appropriately to a water level condition. In the Float Failure State 1408, the noninvasive sensor becomes the sole monitor for determining fluid level and controlling the Submersible Pump 806. The Float Failure State 1408 may trigger a Warning Alerts 642 notification to inform the user that the Float Switch 804 requires inspection or replacement while the system continues to operate using the noninvasive sensor as the sole monitoring mechanism.

A Sensor Failure 1412 state represents the condition where the noninvasive sensor within the Monitoring System 808 has failed while the Float Switch 804 remains operational. The system may transition to the Sensor Failure 1412 state when the noninvasive sensor provides a reading that is statistically improbable or inconsistent with a historical rate of change, indicating that the sensor has malfunctioned rather than detecting an actual change in water level. In certain embodiments, a sensor heartbeat test may be regularly used to determine whether the noninvasive sensor is active. In certain embodiments, the Edge Computing Module 712 maintains a historical record of fluid level measurements and calculates statistical parameters representing expected measurement ranges and rates of change, where a measurement that deviates from the expected range by more than a predefined number of standard deviations or that indicates a rate of change exceeding physical plausibility may trigger detection of sensor failure. In the Sensor Failure 1412 state, the Float Switch 804 takes over as the primary monitor for determining fluid level and controlling the Submersible Pump 806. The Sensor Failure 1412 state may trigger one or more Warning Alerts 642 notifications to inform the user that the Monitoring System 808 requires inspection or replacement while the system continues to operate using the Float Switch 804 as the sole monitoring mechanism.

With continued reference to FIGS. 14A and 14B, an Emergency State 1410 represents the condition where both the noninvasive sensor and the Float Switch 804 have failed, leaving the system without a functioning sensor to monitor fluid level within the sump basin. The system may transition to the Emergency State 1410 from one or more of the Float Failure State 1408 or the Sensor Failure 1412 state when the remaining operational sensor also fails. The Emergency State 1410 may trigger a Critical Interventions 644 alert requiring immediate manual intervention, as the system can no longer automatically monitor fluid level or control the Submersible Pump 806. In certain embodiments, the Emergency State 1410 causes the system to activate the Submersible Pump 806 in a fail-safe mode that operates the pump on a timed cycle until manual intervention restores sensor functionality, thereby providing some protection against flooding even in the absence of functional sensors. In certain embodiments, when an Emergency State 1410 is reached, one or more of a push notification to a user, an SMS or voice call escalation, an email to a maintenance team, or a cloud platform critical (e.g., high priority) log may be triggered.

Though FIG. 14 depicts an electrical sensor as a primary sensor and a float sensor as a backup sensor, in alternative embodiments, a float sensor may serve as the primary sensor and an electrical sensor may serve as the backup sensor. Moreover, any number of sensors may be used without departing from the scope of the present disclosure, including any combination of one or more float sensors and/or one or more electrical sensors.

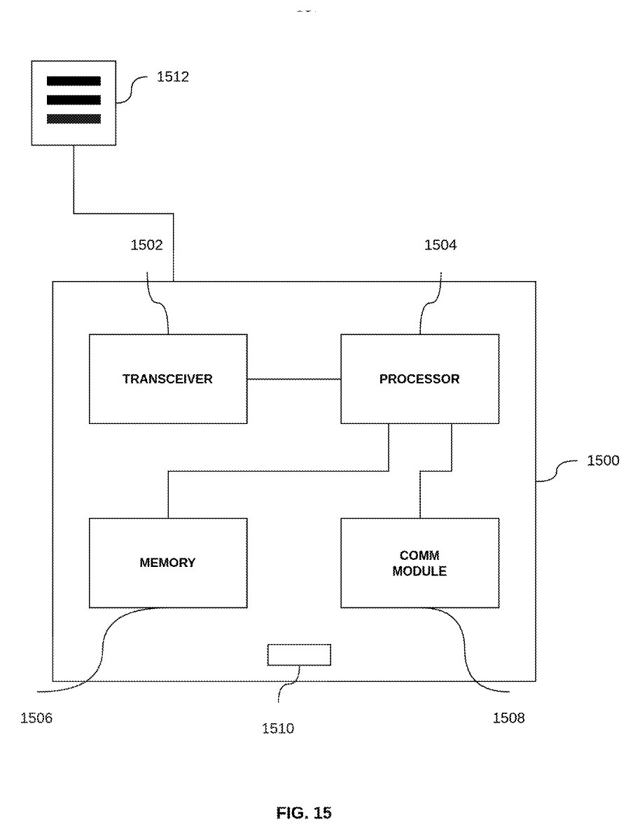

Referring to FIG. 15, processing system 1500 may comprise one or more transceivers 1502, one or more processors 1504, memory 1506, one or more communication modules 1508, power source 1510, and one or more sensors 1512.

Processor 1504 may comprise one or more conventional processing systems (e.g., INTEL Pentium serial processors) that operate to access instructions and provide control instructions to processing system 1500. Alternatively, processor 1504 may comprise dedicated hardware and software that may provide control instructions.

In some aspects, Memory 1506 provides storage capability for instructions (software, code) that may be accessed by processor 1504. Memory 1506 may for example be represented as semiconductor memory, such as a combination of PROM (programmable read- only memory), wherein instructions are permanently stored or RAM (random access memory), wherein data values may be accessed and overwritten.

In some aspects, communication module 1508 is configured to provide data collected by processor 1504 to one or more external devices (not shown), which may be used to evaluate, correlate and collate the data collected. Communication module 1508 may comprise a wired or a wireless communication connection to the not shown external devices. For example, communication module 1508 may be in wired communication with one or more systems that may be in communication with the Internet that allows for the monitoring of the determined fluid level over a broad geographical area.

Alternatively, communication module 1508 may include elements that provide information through one or more wireless communication protocols (e.g., a very short-range NFC protocol (e.g., RFID), a short-range protocol (BLUETOOTH), a longer-range protocol (Wi-Fi) and a long-range protocol (e.g., cellular)). In addition, communication module 1508 may operate to receive information from an external source either through a wired communication protocol or a wireless communication protocol.

In some aspects, Power source 1510 provides power (electrical energy) to the electrical/electronic components of processing system 1500. In one aspect of the invention, power source 1510 may represent a lithium-nickel battery that provides power to processing system 1500 for an extended period of time. In another aspect of the invention, power source 1510 may be a rechargeable battery element that may be recharged by removal from processing system 1500 or recharged while included within processing system 1500. Alternatively, power source 1510 may be an AC to DC converter that receives electrical energy from a main source of power (e.g., 120-volt outlet) and converts the received power to a direct current that is used to power the electrical/electronic components of processing system 1500.

Various techniques, or certain aspects or portions thereof, may take the form of program code (i.e., instructions) embodied in tangible media, such as floppy diskettes, CD- ROMs, hard drives, a non-transitory computer readable storage medium, or any other machine- readable storage medium wherein, when the program code is loaded into and executed by a machine, such as a computer, the machine becomes an apparatus for practicing the various techniques. In the case of program code execution on programmable computers, the computing device may include a processor, a storage medium readable by the processor (including volatile and non-volatile memory and/or storage elements), at least one input device, and at least one output device. The volatile and non-volatile memory and/or storage elements may be a RAM, an EPROM, a flash drive, an optical drive, a magnetic hard drive, or another medium for storing electronic data. The eNB (or other base station) and UE (or other mobile station) may also include a transceiver component, a counter component, a processing component, and/or a clock component or timer component. One or more programs that may implement or utilize the various techniques described herein may use an application programming interface (API), reusable controls, and the like. Such programs may be implemented in a high-level procedural or an object-oriented programming language to communicate with a computer system. However, the program(s) may be implemented in assembly or machine language, if desired. In any case, the language may be a compiled or an interpreted language, and combined with hardware implementations.

It should be understood that many of the functional units described in this specification may be implemented as one or more components, which is a term used to more particularly emphasize their implementation independence. For example, a component may be implemented as a hardware circuit comprising custom very large scale integration (VLSI) circuits or gate arrays, off-the-shelf semiconductors such as logic chips, transistors, or other discrete components. A component may also be implemented in programmable hardware devices such as field programmable gate arrays, programmable array logic, programmable logic devices, or the like.

Components may also be implemented in software for execution by various types of processors. An identified component of executable code may, for instance, comprise one or more physical or logical blocks of computer instructions, which may, for instance, be organized as an object, a procedure, or a function. Nevertheless, the executables of an identified component need not be physically located together, but may comprise disparate instructions stored in different locations that, when joined logically together, comprise the component and achieve the stated purpose for the component.

Indeed, a component of executable code may be a single instruction, or many instructions, and may even be distributed over several different code segments, among different programs, and across several memory devices. Similarly, operational data may be identified and illustrated herein within components, and may be embodied in any suitable form and organized within any suitable type of data structure. The operational data may be collected as a single data set, or may be distributed over different locations including over different storage devices, and may exist, at least partially, merely as electronic signals on a system or network. The components may be passive or active, including agents operable to perform desired functions.

Reference throughout this specification to "an example" means that a particular feature, structure, or characteristic described in connection with the example is included in at least one embodiment of the present invention. Thus, appearances of the phrase "in an example" in various places throughout this specification are not necessarily all referring to the same embodiment.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on its presentation in a common group without indications to the contrary. In addition, various embodiments and examples of the present invention may be referred to herein along with alternatives for the various components thereof. It is understood that such embodiments, examples, and alternatives are not to be construed as de facto equivalents of one another, but are to be considered as separate and autonomous representations of the present invention.

As used throughout this disclosure, the phrase "one or more of' followed by a list of items separated by "or" (e.g., "one or more of A, B, or C") is intended to mean one or more items selected from the list, including any single item alone, any combination of items, or all items together, as well as any combination with multiples of the same element. For example, "one or more of A, B, or C" includes: A alone; B alone; C alone; A and B together; A and C together; B and C together; or A, B, and C together. This interpretation applies equally to the phrase "at least one of' followed by a list of items. As an example, "at least one of: a, b, or c" is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). This clarification is provided to ensure consistent interpretation of claim scope and is not intended to limit the disclosure in any way.

The invention has been described with reference to specific embodiments. One of ordinary skill in the art, however, appreciates that various modifications and changes can be made without departing from the scope of the invention as set forth in the claims. Accordingly, the specification is to be regarded in an illustrative manner, rather than with a restrictive view, and all such modifications are intended to be included within the scope of the invention.

Benefits, other advantages, and solutions to problems have been described above regarding specific embodiments. The benefits, advantages, and solutions to problems, and any element(s) that may cause any benefits, advantages, or solutions to occur or become more pronounced, are not to be construed as a critical, required, or an essential feature or element of any or all of the claims.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the term "determining" encompasses a wide variety of actions. For example, "determining" may include calculating, computing, processing, deriving, investigating, looking up (e.g., looking up in a table, a database or another data structure), ascertaining and the like. Also, "determining" may include receiving (e.g., receiving information), accessing (e.g., accessing data in a memory) and the like. Also, "determining" may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Although the foregoing has been described in some detail for purposes of clarity, it will be apparent that certain changes and modifications may be made without departing from the principles thereof. It should be noted that there are many alternative ways of implementing both the processes and apparatuses described herein. Accordingly, the present embodiments are to be considered illustrative and not restrictive, and the invention is not to be limited to the details given herein, but may be modified within the scope and equivalents of the appended claims.

The following claims are not intended to be limited to the embodiments shown herein, but are to be accorded the full scope consistent with the language of the claims. Within a claim, reference to an element in the singular is not intended to mean "one and only one" unless specifically so stated, but rather "one or more." Unless specifically stated otherwise, the term "some" refers to one or more. No claim element is to be construed under the provisions of 35 U.S.C. § 112(f) unless the element is expressly recited using the phrase "means for" or, in the case of a method claim, the element is recited using the phrase "step for." All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A method for creating a tamper-proof audit trail for container monitoring data, comprising:
acquiring sensor data from a noninvasive sensor monitoring a container;
generating a cryptographic hash of the sensor data at an edge computing device;
transmitting the sensor data and the cryptographic hash to a remote server over an encrypted communication channel;
processing the sensor data using a machine learning model to generate a prediction regarding contents of the container; and
recording the cryptographic hash on a distributed blockchain ledger to create an immutable record of the sensor data.

2. The method of claim 1, wherein the encrypted communication channel comprises a Transport Layer Security (TLS) encrypted channel.

3. The method of claim 1, further comprising:
generating an alert based on the prediction, wherein the alert includes forensic metadata comprising the sensor data, the prediction, a confidence score, and a timestamp.

4. The method of claim 3, wherein the alert is classified into one of a plurality of severity tiers comprising an informational notification tier, a warning alert tier, and a critical intervention tier based on a magnitude of a detected deviation from an expected condition.

5. The method of claim 1, further comprising:
automatically executing a smart contract deployed on the distributed blockchain ledger based on the recorded cryptographic hash, wherein the smart contract triggers a predefined action when a condition defined in the smart contract is met by the prediction.

6. The method of claim 5, wherein the predefined action comprises an automated insurance payout triggered by detection of a contamination event indicated by the prediction.

7. The method of claim 1, further comprising:
generating a compliance report based on the prediction and the immutable record on the distributed blockchain ledger, wherein the compliance report is formatted for submission to a regulatory authority.

8. The method of claim 1, wherein the sensor data comprises at least one of a dielectric property measurement across a plurality of frequencies or a fluid level measurement derived from radar return signals.

9. The method of claim 8, further comprising:
constructing a dielectric spectrum based on the dielectric property measurement across the plurality of frequencies;
comparing the dielectric spectrum to a baseline dielectric fingerprint; and
detecting a contamination event based on a deviation between the dielectric spectrum and the baseline dielectric fingerprint exceeding a predefined threshold.

10. The method of claim 1, further comprising:
analyzing a time-series of sensor data collected over a period of time to determine a rate of change of a measured property; and
predicting a future contamination level or a time-to-failure based on the rate of change.

11. A non-transitory computer-readable medium storing instructions that, when executed by a processor, cause the processor to perform operations comprising:
acquiring sensor data from a noninvasive sensor monitoring a container;
generating a cryptographic hash of the sensor data at an edge computing device;
transmitting the sensor data and the cryptographic hash to a remote server over an encrypted communication channel;
processing the sensor data using a machine learning model to generate a prediction regarding contents of the container; and
recording the cryptographic hash on a distributed blockchain ledger to create an immutable record of the sensor data.

12. The non-transitory computer-readable medium of claim 11, wherein the encrypted communication channel comprises a Transport Layer Security (TLS) encrypted channel.

13. The non-transitory computer-readable medium of claim 11, further comprising:
generating an alert based on the prediction, wherein the alert includes forensic metadata comprising the sensor data, the prediction, a confidence score, and a timestamp.

14. The non-transitory computer-readable medium of claim 13, wherein the alert is classified into one of a plurality of severity tiers comprising an informational notification tier, a warning alert tier, and a critical intervention tier based on a magnitude of a detected deviation from an expected condition.

15. The non-transitory computer-readable medium of claim 11, further comprising:
automatically executing a smart contract deployed on the distributed blockchain ledger based on the recorded cryptographic hash, wherein the smart contract triggers a predefined action when a condition defined in the smart contract is met by the prediction.

16. The non-transitory computer-readable medium of claim 15, wherein the predefined action comprises an automated insurance payout triggered by detection of a contamination event indicated by the prediction.

17. The non-transitory computer-readable medium of claim 11, further comprising:
generating a compliance report based on the prediction and the immutable record on the distributed blockchain ledger, wherein the compliance report is formatted for submission to a regulatory authority.

18. The non-transitory computer-readable medium of claim 11, wherein the sensor data comprises at least one of a dielectric property measurement across a plurality of frequencies or a fluid level measurement derived from radar return signals.

19. The non-transitory computer-readable medium of claim 18, further comprising:
constructing a dielectric spectrum based on the dielectric property measurement across the plurality of frequencies;
comparing the dielectric spectrum to a baseline dielectric fingerprint; and
detecting a contamination event based on a deviation between the dielectric spectrum and the baseline dielectric fingerprint exceeding a predefined threshold.

20. The non-transitory computer-readable medium of claim 11, further comprising:
analyzing a time-series of sensor data collected over a period of time to determine a rate of change of a measured property; and
predicting a future contamination level or a time-to-failure based on the rate of change.

21. A system for creating a tamper-proof audit trail for container monitoring data, comprising:
a noninvasive sensor configured for external mounting to a container; and
one or more processors configured to perform operations comprising:
acquiring sensor data from the noninvasive sensor monitoring the container;
generating a cryptographic hash of the sensor data at an edge computing device;
transmitting the sensor data and the cryptographic hash to a remote server over an encrypted communication channel;
processing the sensor data using a machine learning model to generate a prediction regarding contents of the container; and
recording the cryptographic hash on a distributed blockchain ledger to create an immutable record of the sensor data.

22. The system of claim 21, wherein the encrypted communication channel comprises a Transport Layer Security (TLS) encrypted channel.

23. The system of claim 21, further comprising:
generating an alert based on the prediction, wherein the alert includes forensic metadata comprising the sensor data, the prediction, a confidence score, and a timestamp.

24. The system of claim 23, wherein the alert is classified into one of a plurality of severity tiers comprising an informational notification tier, a warning alert tier, and a critical intervention tier based on a magnitude of a detected deviation from an expected condition.

25. The system of claim 21, further comprising:
automatically executing a smart contract deployed on the distributed blockchain ledger based on the recorded cryptographic hash, wherein the smart contract triggers a predefined action when a condition defined in the smart contract is met by the prediction.

26. The system of claim 25, wherein the predefined action comprises an automated insurance payout triggered by detection of a contamination event indicated by the prediction.

27. The system of claim 21, further comprising:
generating a compliance report based on the prediction and the immutable record on the distributed blockchain ledger, wherein the compliance report is formatted for submission to a regulatory authority.

28. The system of claim 21, wherein the sensor data comprises at least one of a dielectric property measurement across a plurality of frequencies or a fluid level measurement derived from radar return signals.

29. The system of claim 28, further comprising:
constructing a dielectric spectrum based on the dielectric property measurement across the plurality of frequencies;
comparing the dielectric spectrum to a baseline dielectric fingerprint; and
detecting a contamination event based on a deviation between the dielectric spectrum and the baseline dielectric fingerprint exceeding a predefined threshold.

30. The system of claim 21, further comprising:
analyzing a time-series of sensor data collected over a period of time to determine a rate of change of a measured property; and
predicting a future contamination level or a time-to-failure based on the rate of change.
